(19) United States
(12) Patent Application Publication
(10) Pub. No.: US 2026/0287496 A1
(43) Pub. Date: Sep. 24, 2026

(54) OPTICAL CONCENTRATION MEASURING DEVICE

(71) Applicant: Asahi Kasei Microdevices Corporation, Tokyo (JP)

(72) Inventors: Tatsushi YAGI, Tokyo (JP); Naoya IWATA, Tokyo (JP)

(21) Appl. No.: 19/549,014

(22) Filed: Feb. 25, 2026

(30) Foreign Application Priority Data
Mar. 21, 2025 (JP) 2025-046301

(51) Int. Cl. G01N 21/17; G01N 33/00

(52) U.S. Cl. CPC G01N 21/1702; G01N 2021/1704; G01N 33/0009; G01N 2201/067

(57) ABSTRACT

Provided is an optical concentration measuring device for measuring a concentration of a target component in a gas, comprising an acoustic cavity that has a gas inlet opening and contains the gas introduced therein via the gas inlet opening, an optical part installed within the acoustic cavity, and an acoustic transducer installed within the acoustic cavity, wherein the optical part includes one or more of each of a light source that emits light including a wavelength that is absorbed by the target component and an optical waveguide which is optically connected to the light source and inside which the light from the light source propagates and at least a portion of which is exposed to a space within the acoustic cavity, and the optical part has a plurality of optical waveguides, each being identical to the optical waveguide, that are optically connected to the light source.

The contents of the following patent application(s) are incorporated herein by reference:
NO. 2025-046301 filed in JP on Mar. 21, 2025.

### BACKGROUND

### 1. Technical Field

The present invention relates to an optical concentration measuring device.

### 2. Related Art

Patent document 1 describes that “The proposed photoacoustic gas sensor device, as e.g. shown in FIG. 1, may be built with a small form factor, such that it has an overall size of e.g. 1×1×0.7 cm³” (0040).

### RELATED ART DOCUMENTS

### Patent Documents

Patent Document 1: US2022/0187193 A1

### BRIEF DESCRIPTION OF THE DRAWINGS

FIG. 1 is a schematic side view of an optical concentration measuring device 100 according to an implementation.

FIG. 2 is a schematic plan view of the optical concentration measuring device 100 shown in FIG. 1.

FIG. 3 is a schematic side view of an optical part 220 according to an implementation.

FIG. 4 is a schematic side view of an optical concentration measuring device 300 according to an implementation.

FIG. 5 is a schematic side view of an optical part 321 according to an implementation.

FIG. 6 is a schematic side view of an optical part 322 according to an implementation.

FIG. 7 is a schematic side view of an optical part 323 according to an implementation.

FIG. 8 is a schematic side view of an optical concentration measuring device 400 according to an implementation.

FIG. 9 is a schematic side view of an optical concentration measuring device 401 according to an implementation.

FIG. 10 is a schematic side view of an optical concentration measuring device 402 according to an implementation.

FIG. 11 is a schematic side view of an optical concentration measuring device 500 according to an implementation.

FIG. 12 is a schematic side view of an optical concentration measuring device 600 according to an implementation.

FIG. 13 is a schematic side view of an optical part 621 according to an implementation.

FIG. 14 is a schematic side view of an optical part 622 according to an implementation.

FIG. 15 is a schematic plan view of an optical waveguide 741 according to an implementation.

FIG. 16 is a schematic plan view of an optical waveguide 742 according to an implementation.

FIG. 17 is a schematic plan view of an optical waveguide 743 according to an implementation.

FIG. 18 is a schematic side view of an optical waveguide 840 of an optical part 820 and a light source 130 according to an implementation.

FIG. 19 is a schematic plan view of an optical part 920 according to an implementation.

FIG. 20 is a schematic plan view of an optical part 921 according to an implementation.

FIG. 21 is a schematic side view of the optical part 921 shown in FIG. 20.

FIG. 22 is a schematic plan view of an optical part 1021 according to an implementation.

FIG. 23 is a schematic plan view of an optical waveguide 1140 of an optical part 1120 and the light source 130 according to an implementation.

FIG. 24 is a schematic plan view of an optical waveguide 1240 of an optical part 1220 and the light source 130 according to an implementation.

FIG. 25 is a schematic plan view of an optical concentration measuring device 1300 according to an implementation.

FIG. 26 is a schematic side view of the optical concentration measuring device 1300 shown in FIG. 25.

FIG. 27 is a schematic plan view of an optical concentration measuring device 1400 according to an implementation.

FIG. 28 is a schematic side view of the optical concentration measuring device 1400 shown in FIG. 27.

FIG. 29 is a schematic plan view of an optical part 1520 according to an implementation.

FIG. 30 is a schematic plan view of an optical part 1620 according to an implementation.

FIG. 31 is a schematic side view of the optical part 1620 shown in FIG. 30.

FIG. 32 is a schematic plan view of an optical concentration measuring device 1800 according to an implementation.

FIG. 33 is a schematic plan view of an optical filter. 1881, a light source 1831, and an optical waveguide 140 of an optical part 1821 according to an implementation.

FIG. 34 is a schematic plan view of an optical filter. 1882, a light source 1832, and an optical waveguide 140 of an optical part 1822 according to an implementation.

FIG. 35 is a schematic plan view of an optical filter. 1883, a light source 1833, and an optical waveguide 140 of an optical part 1823 according to an implementation.

FIG. 36 is a schematic plan view of an optical concentration measuring device 1900 according to an implementation.

FIG. 37 is a schematic plan view of an optical concentration measuring device 1901 according to an implementation.

FIG. 38 is a schematic plan view of an optical concentration measuring device 2000 according to an implementation.

FIG. 39 is a schematic plan view of an optical concentration measuring device 2001 according to an implementation.

FIG. 40 is a schematic side view of the optical concentration measuring device 600 shown in FIG. 6.

FIG. 41 is a schematic side view of an optical concentration measuring device 2100 according to an implementation.

FIG. 42 is a schematic side view of an optical concentration measuring device 2100 according to an implementation.

FIG. 43 is a schematic side view of an optical concentration measuring device 2200 according to an implementation.

FIG. 44 is a schematic side view of an optical concentration measuring device 2300 according to an implementation.

FIG. 45 is a schematic plan view of an optical concentration measuring device 2400 according to an implementation.

FIG. 46 illustrates an example of a method of calculating a total length of the optical waveguide 743 shown in FIG. 17, which has a circulating shape with a curve in a plan view.

FIG. 47 is a graph representing a relationship between a length of one side of a square surrounding the optical waveguide 743 in the plan view and each of the total length and an effective optical path length of the optical waveguide 743.

FIG. 48 is a graph representing a relationship between a length of one side of an occupied area of the square and the effective optical path length of the optical waveguide 743, according to the optical concentration measuring device 600 or the like including the optical waveguide 743.

FIG. 49 is a graph representing a relationship between a volume of the optical concentration measuring device 600 or the like and the effective optical path length of the optical waveguide 743 in the example of FIG. 48.

### DESCRIPTION OF EXEMPLARY EMBODIMENTS

Embodiments described below do not limit the invention according to the claims. Not all combinations of features described in the embodiments are necessarily essential for a solution of the invention.

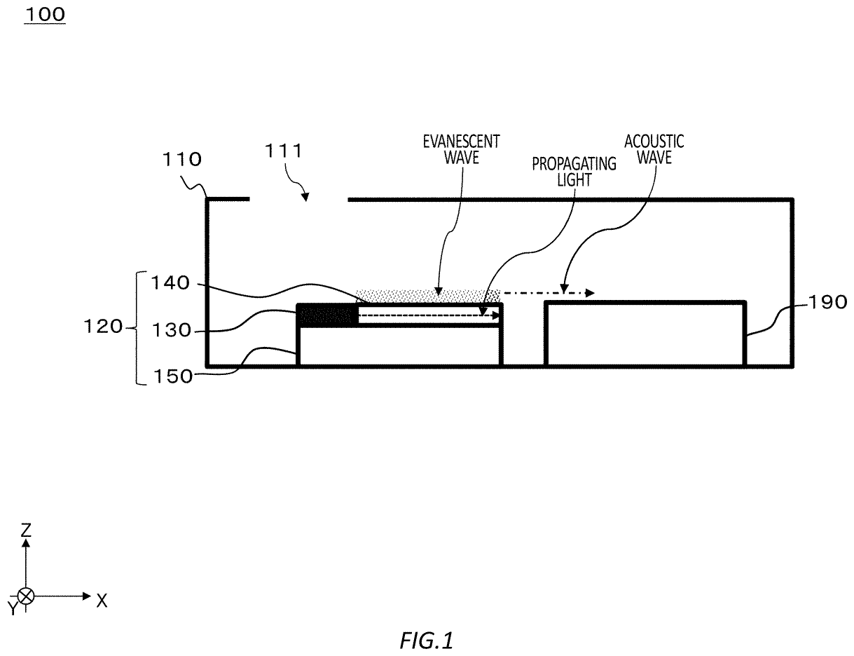

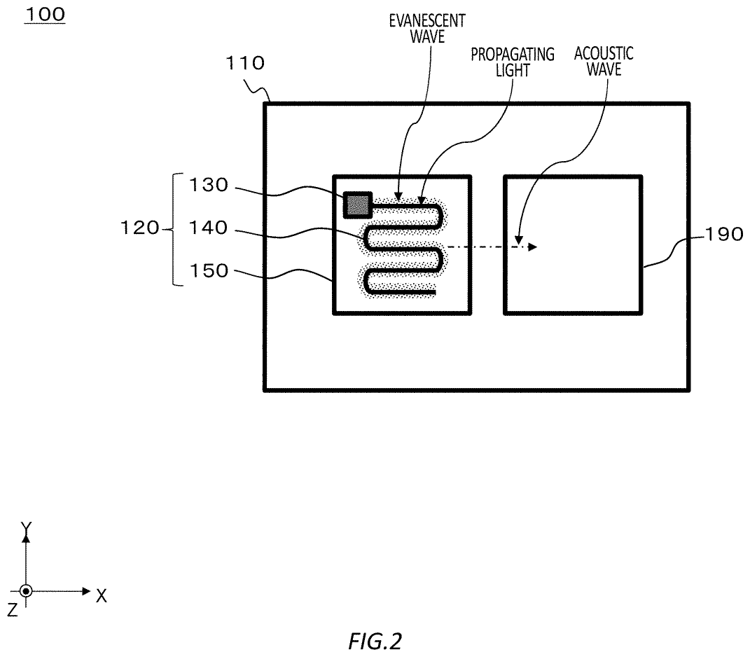

FIG. 1 is a schematic side view of an optical concentration measuring device 100 according to an implementation. FIG. 2 is a schematic plan view of the optical concentration measuring device 100 shown in FIG. 1. X, Y, and Z axes, which are orthogonal to each other, are shown in FIG. 1. In FIG. 1, the X axis is an axis that extends in the left-right direction facing a sheet of paper, with the right side of the sheet of paper being the positive side of the X axis. The Y axis is an axis that extends in a depth direction toward the sheet of paper, with the deeper side of the sheet of paper being the positive side of the Y axis. The Z axis is an axis that extends in the up-down direction facing the sheet of paper, with the top side of the sheet of paper being the positive side of the Z axis. X, Y, and Z axes corresponding to the X, Y, and Z axes in FIG. 1 are shown for each figure beyond FIG. 2, and duplicate descriptions will be omitted thereafter.

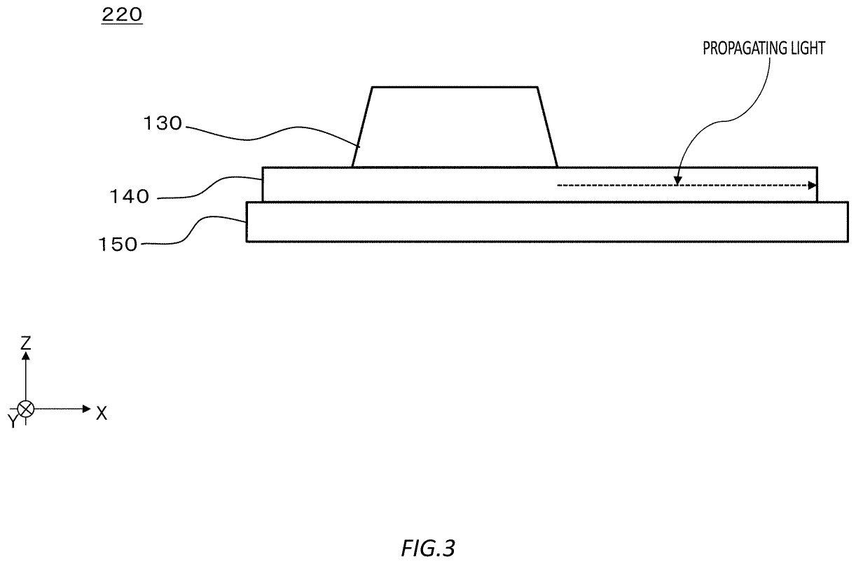

The optical concentration measuring device 100 includes an acoustic cavity 110 that contains gas, and an optical part 120 and an acoustic transducer 190 installed within the acoustic cavity 110. In the side view in FIG. 1 and the plan view in FIG. 2, a portion of the acoustic cavity 110 is shown in a transparent manner. The same applies to each of the side views and plan views beyond FIG. 3, and duplicate descriptions will be omitted thereafter.

The optical concentration measuring device 100 utilizes photoacoustic effects to measure a concentration of a target component in the gas contained within the acoustic cavity 110. The photoacoustic effect is a phenomenon in which acoustic waves are generated through volume expansion caused by heat released by a molecule that absorbed light energy. The optical concentration measuring device 100 may also be referred to as a photoacoustic gas sensor.

The acoustic cavity 110 has a gas inlet opening 111, and contains gas introduced via the gas inlet opening 111. An inner wall of the acoustic cavity 110 may or may not reflect light.

The optical part 120 has a light source 130, an optical waveguide 140, and a substrate 150. The optical part 120 may have one or more of each of the light source 130, the optical waveguide 140, and the substrate 150; in the present example, the optical part 120 has one of each.

The light source 130 emits light including a wavelength that is absorbed by the target component in the gas. The wavelength of the light emitted by the light source 130 may be set according to the target component for which the optical concentration measuring device 100 measures the concentration. For example, when the target component is a carbon dioxide molecule (CO₂), the wavelength of said light may be approximately a few μm, that is, said light may be mid infrared rays. Note that, in each figure beyond FIG. 1, the light from the light source 130 is schematically illustrated with a straight dashed line, and duplicate descriptions will be omitted thereafter.

The optical waveguide 140 is optically connected to the light source 130 and the light from the light source 130 propagates therein. At least a portion of the optical waveguide 140 is exposed in a space within the acoustic cavity 110. The total length of the optical waveguide 140 is, for example, approximately 2 cm. When the optical part 120 has a plurality of optical waveguides 140, a sum of the total length of the plurality of optical waveguides 140 may be approximately 2 cm. The width of the optical waveguide 140 is, for example, approximately 3 μm. These dimensions may vary according to the design of the optical concentration measuring device 100, the optical waveguide 140 or the like.

The substrate 150 supports the light source 130 and the optical waveguide 140. In the present example, the light source 130 and the optical waveguide 140 are provided together on one substrate 150, and are in direct contact with each other.

The optical concentration measuring device 100 may cause the acoustic transducer 190 to detect acoustic waves generated due to absorption, by the target component, of light from the optical part 120, and measure the concentration of the target component based on the intensity of an electrical signal output from the acoustic transducer 190 that detected said acoustic waves. More specifically, the acoustic transducer 190 outputs an electrical signal according to a pressure fluctuation within the acoustic cavity 110 that is generated by causing the target component included in a gas that is present around the optical waveguide 140 and having a lower refractive index than the optical waveguide 140, to absorb an evanescent wave that leaks to the outside of the optical waveguide 140 during propagation of the light from the light source 130 within the optical waveguide 140. Note that, said evanescent wave is schematically illustrated with dots in each figure beyond FIG. 1, said acoustic wave is schematically illustrated with a straight dot and dash line, and duplicate descriptions will be omitted thereafter.

By separating an optical path of the light to be absorbed by the target component in the gas from the acoustic cavity 110, which contains the gas and in which a sound generated due to absorption of the light by the target component resonates, the optical concentration measuring device 100 has a reduced volume, that is, it can be made more compact, compared to a measurement device of a comparative example in which the optical path and the acoustic cavity 110 are not separated. As an example, the optical concentration measuring device 100 has a volume of 10 mm³ or less. The optical concentration measuring device 100 is thus made more compact, while also securing an effective optical path length of the optical waveguide 140 of 10 mm or more. The relationship between the volume and the effective optical path length of the optical concentration measuring device 100 will be described in detail below.

In addition, by separating the optical path from the acoustic cavity 110, the optical concentration measuring device 100 has an improved degree of freedom in the design and manufacture of the optical concentration measuring device 100 compared to the measurement device of the comparative example described above. Note that, the measurement device of the comparative example described above may be the measurement device disclosed in Patent document 1 described above, for example.

Note that, although in each figure beyond FIG. 1, the optical part 120 and the acoustic transducer 190 of the optical concentration measuring device 100 are illustrated as having similar dimensions to each other merely for the purpose of clarifying the description, the dimensional ratio of these components is not to be limited to the illustrated dimensional ratio in any way. Note that, in the optical concentration measuring device 100, the optical part 120 may not have the substrate 150. In this case, the light source 130 and the optical waveguide 140 of the optical part 120 may be directly installed within the acoustic cavity 110.

FIG. 3 is a schematic side view of an optical part 220 according to an implementation. The optical concentration measuring device 100 according to an implementation shown in FIG. 1 and FIG. 2 may include, instead of the optical part 120, an optical part 220 in which the light source 130 is formed on the positive side of the Z axis of the optical waveguide 140. In the optical part 220, similarly to the optical part 120, the light source 130 and the optical waveguide 140 are also provided together on one substrate 150, and are in direct contact with each other. By including the optical parts 120, 220 thus configured, the optical concentration measuring device 100 can cause the light from the light source 130 to efficiently propagate through the optical waveguide 140 and can make the optical parts 120, 220 more compact.

Note that, for a plurality of examples described by using a plurality of figures beyond FIG. 3, only differences thereof from an implementation described by using FIG. 1 and FIG. 2 will be described. For a plurality of examples hereinafter, components corresponding to components in an implementation of FIG. 1 and FIG. 2 are provided with similar reference numerals, and duplicate descriptions will be omitted.

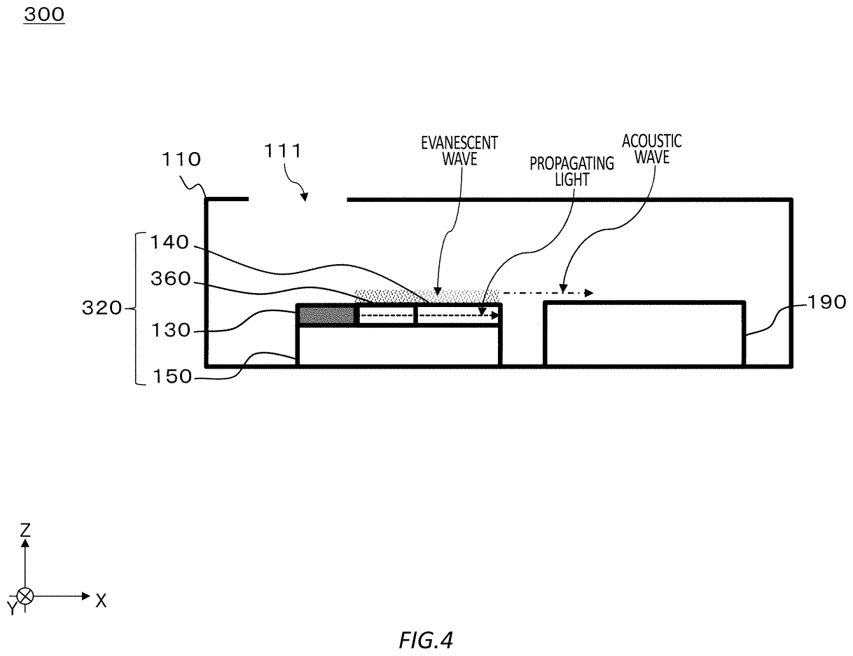

FIG. 4 is a schematic side view of an optical concentration measuring device 300 according to an implementation. As a difference from the optical concentration measuring device 100, the optical concentration measuring device 300 includes, instead of the optical part 120, an optical part 320 having a photocoupler 360. In the optical part 320, the light source 130 and the optical waveguide 140 are provided together on one substrate 150, and are optically coupled to each other via the photocoupler 360. By including the optical part 320 thus configured, the optical concentration measuring device 300 enables each of the light source 130, the photocoupler 360, and the optical waveguide 140 to be independently designed, thereby enhancing the degree of freedom of the design. In addition, by including the optical part 320 thus configured, the optical concentration measuring device 300 can make the optical part 320 more compact.

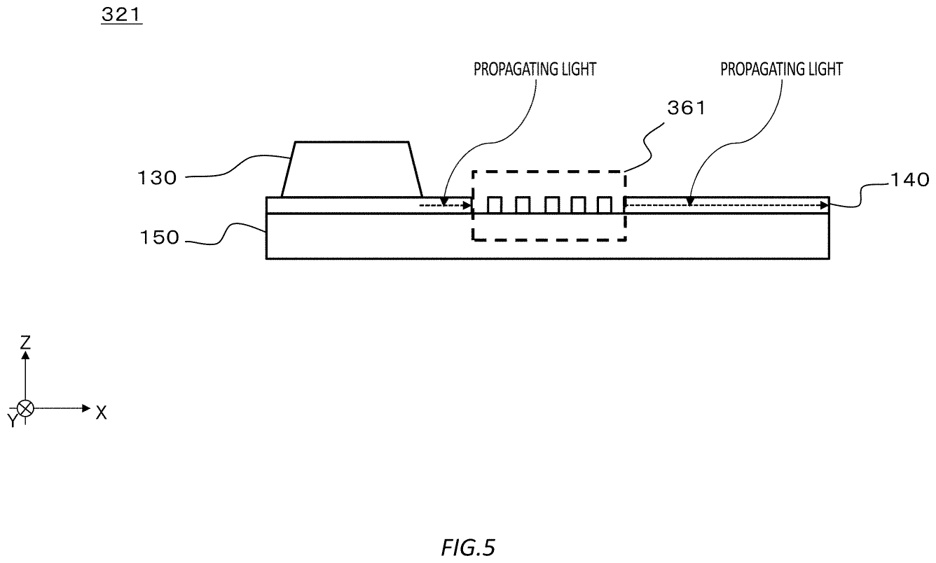

FIG. 5 is a schematic side view of an optical part 321 according to an implementation. The optical concentration measuring device 300 may include, instead of the optical part 320, an optical part 321 having a photocoupler 361 consisting of a grating surrounded by a dashed frame in FIG. 5, that is, a diffraction grating.

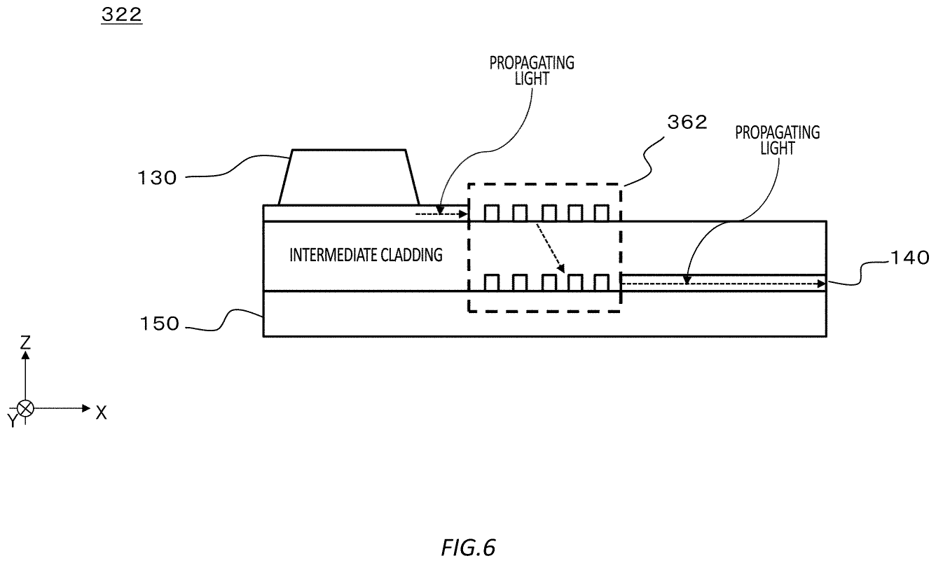

FIG. 6 is a schematic side view of an optical part 322 according to an implementation. The optical concentration measuring device 300 may include, instead of the optical part 320, an optical part 322 having a photocoupler 362, which is surrounded by a dashed frame in FIG. 6, consisting of two sets of gratings with an intermediate cladding interposed therebetween.

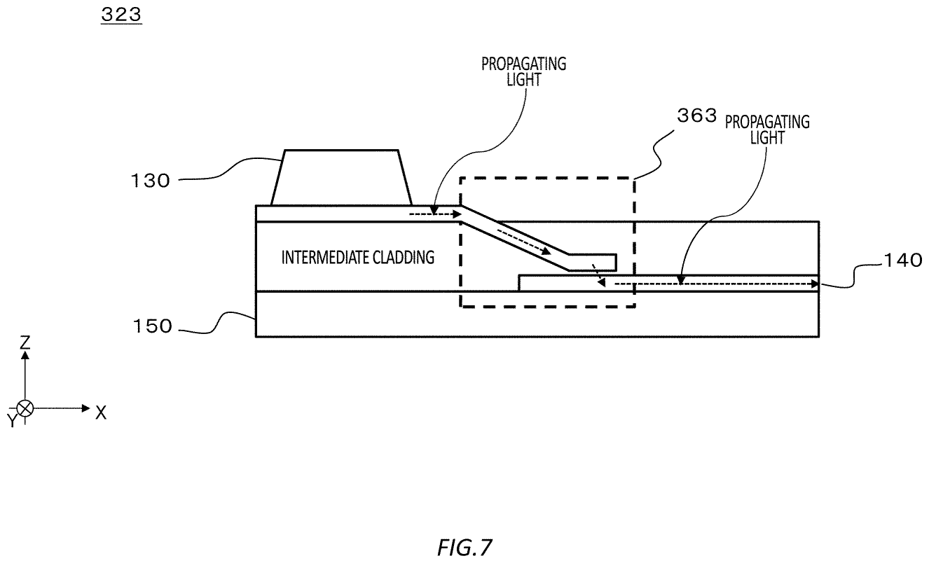

FIG. 7 is a schematic side view of an optical part 323 according to an implementation. The optical concentration measuring device 300 may include, instead of the optical part 320, an optical part 323 having a photocoupler 363, which is surrounded by a dashed frame in FIG. 7, consisting of directional couplers with an intermediate cladding interposed therebetween.

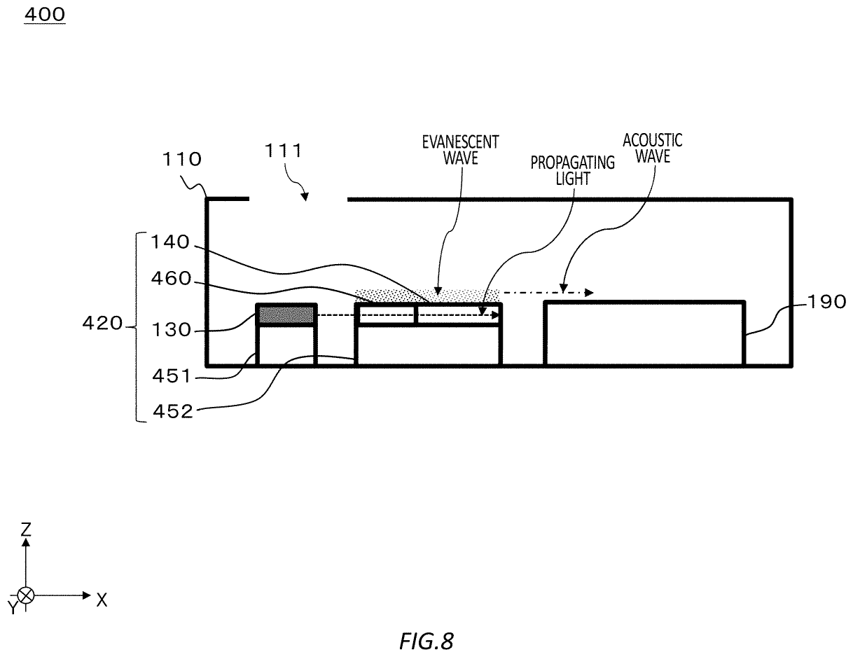

FIG. 8 is a schematic side view of an optical concentration measuring device 400 according to an implementation. As a difference from the optical concentration measuring device 100, the optical concentration measuring device 400 includes, instead of the optical part 120, an optical part 420 having two substrates 451, 452, and a photocoupler 460. In the optical part 420, the light source 130 and the optical waveguide 140 are respectively provided on separate substrates 451, 452, and are optically coupled to each other via the photocoupler 460. By including the optical part 420 thus configured, the optical concentration measuring device 400 enables only the light source 130 to be designed differently from other members such as the photocoupler 460 and the optical waveguide 140, thereby enhancing the degree of freedom of the design.

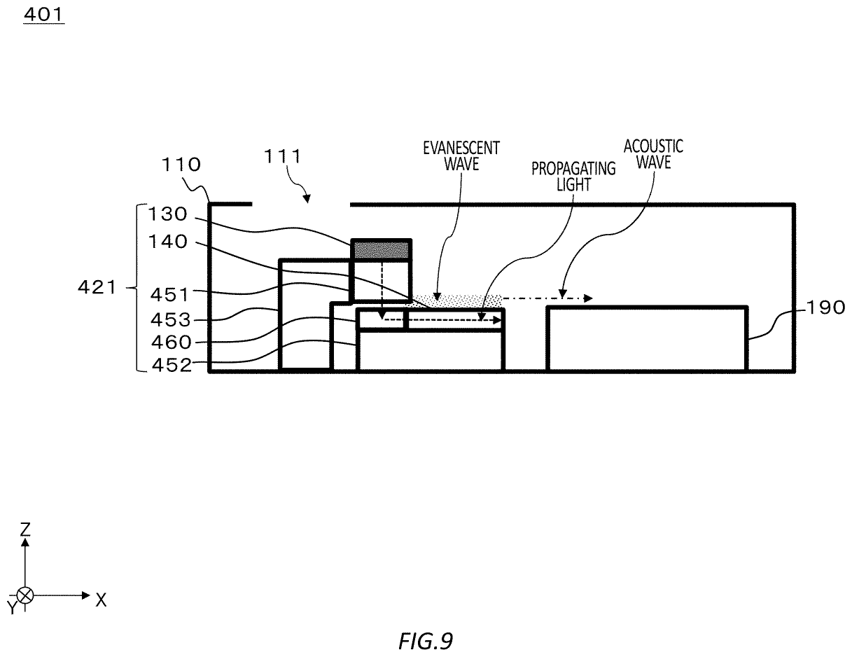

FIG. 9 is a schematic side view of an optical concentration measuring device 401 according to an implementation. As a difference from the optical concentration measuring device 400, the optical concentration measuring device 401 includes, instead of the optical part 420, an optical part 421. In the optical part 420, the light from the light source 130 is emitted from the side surface on the positive side of the x axis of the light source 130, whereas in the optical part 421, the light from the light source 130 is emitted from a surface on the negative side of the Z axis of the substrate 451. The optical part 421 has a substrate support 453 that holds the substrate 451 at a predetermined location.

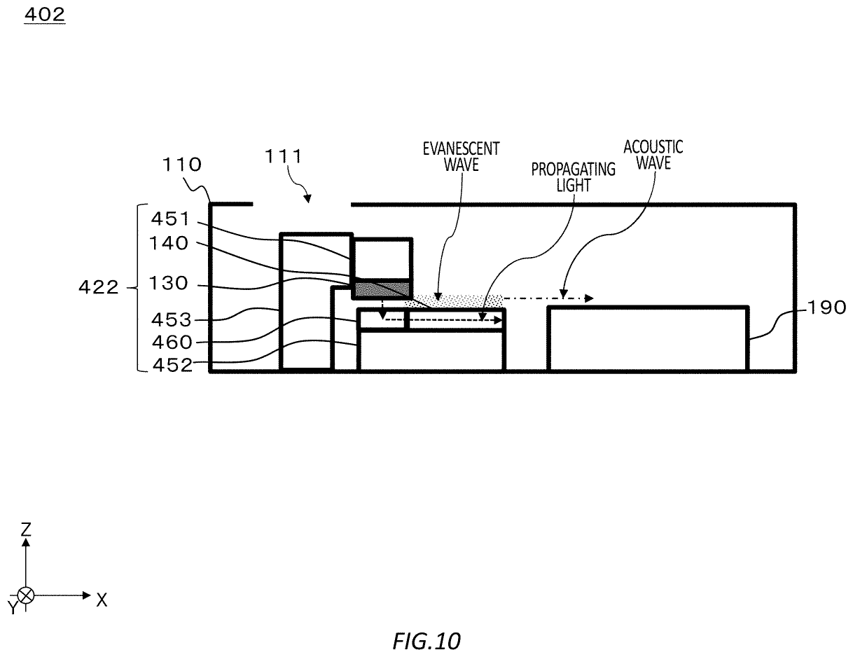

FIG. 10 is a schematic side view of an optical concentration measuring device 402 according to an implementation. As a difference from the optical concentration measuring device 401, the optical concentration measuring device 402 includes, instead of the optical part 421, an optical part 422. In the optical part 421, the light from the light source 130 is emitted from a surface on the negative side of the Z axis of the substrate 451, whereas in the optical part 422, the light from the light source 130 is emitted from a surface on the negative side of the Z axis of the light source 130.

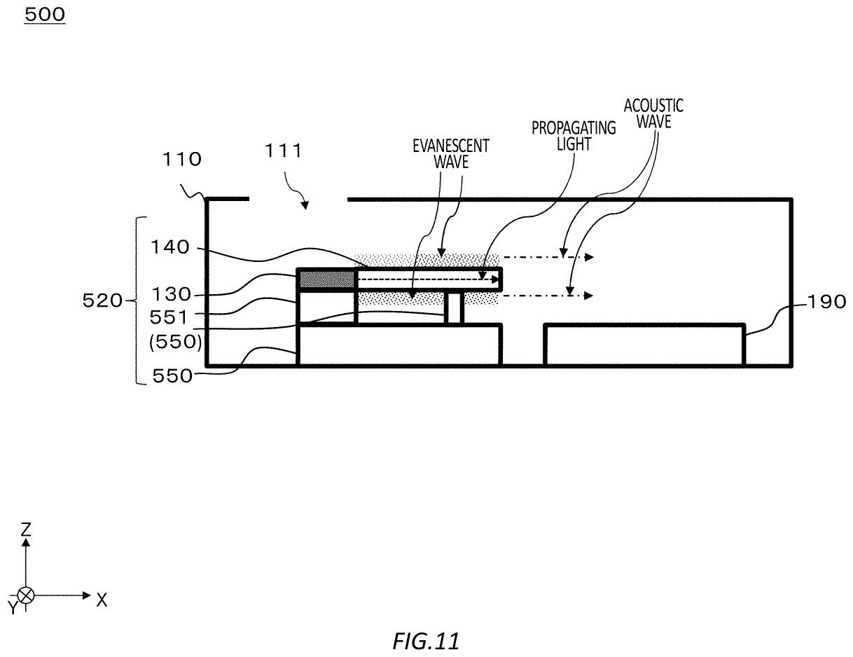

FIG. 11 is a schematic side view of an optical concentration measuring device 500 according to an implementation. As a difference from the optical concentration measuring device 100, the optical concentration measuring device 500 includes, instead of the optical part 120, an optical part 520 in which at least a portion of the optical waveguide 140 is spaced apart from a substrate 550 by a supporting portion 551 of the substrate 550. In the optical part 520, the entire surface of a section of at least a portion of the optical waveguide 140 in the propagation direction of the light that propagates within the optical waveguide 140 is exposed to a space within the acoustic cavity 110 without being in contact with the substrate 550. In the optical part 520, at least a portion of the optical waveguide 140 may be defined as being floating.

By including the optical part 520 thus configured, the optical concentration measuring device 500 can prevent light leakage from the optical waveguide 140 to the substrate 550 or light absorption, thereby enhancing light propagation efficiency at the optical waveguide 140. In order to achieve this effect, a separation distance between the optical waveguide 140 and the substrate 550 may be larger than a leakage length of the evanescent wave that leaks to the outside of the optical waveguide 140 during propagation of the light from the light source 130 within the optical waveguide 140, and may be 2 μm or more, for example. In addition, the optical concentration measuring device 500 including the optical part 520 can increase contact between the evanescent wave that leaks to the outside of the optical waveguide 140 during propagation of the light from the light source 130 within the optical waveguide 140 and the target component included in a gas that is present around the optical waveguide 140 and having a lower refractive index than the optical waveguide 140. The optical concentration measuring device 500 can thereby cause more target components to absorb the evanescent wave to increase the pressure fluctuation width within the acoustic cavity 110, and can increase an output of the acoustic transducer 190.

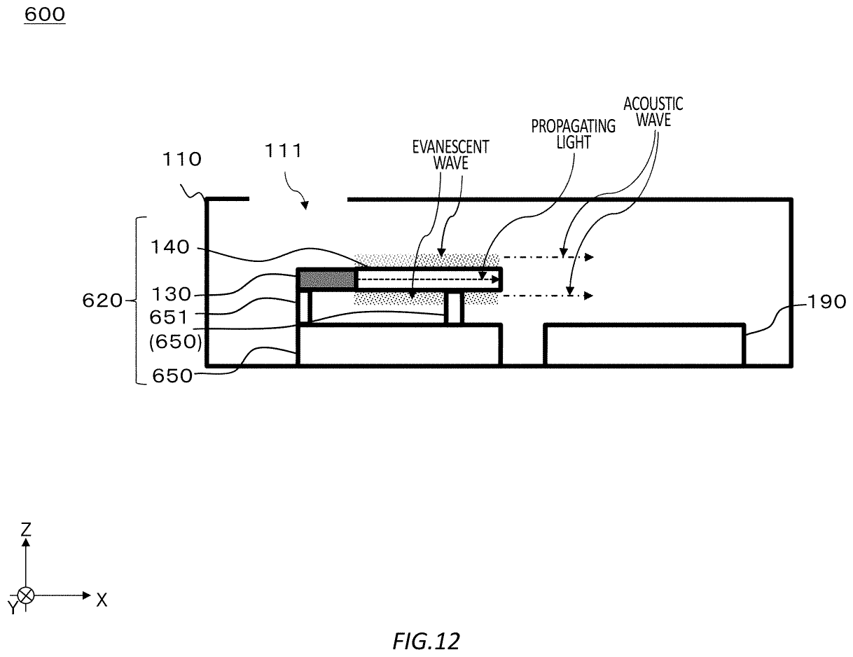

FIG. 12 is a schematic side view of an optical concentration measuring device 600 according to an implementation. As a difference from the optical concentration measuring device 500, the optical concentration measuring device 600 includes, instead of the optical part 520, an optical part 620 in which at least a portion of each of the light source 130 and the optical waveguide 140 is spaced apart from a substrate 650 by the supporting portion 651 of the substrate 650. In the optical part 620, a space is provided between the light source 130 and the substrate 650. In the optical part 620, at least a portion of each of the light source 130 and the optical waveguide 140 may be defined as being floating.

By including the optical part 620 thus configured, the optical concentration measuring device 600 can prevent light leakage from the light source 130 and the optical waveguide 140 to the substrate 650 and light absorption, and can cause the light to be efficiently propagated from the light source 130 to the optical waveguide 140 and enhance the light propagation efficiency at the optical waveguide 140. In order to achieve this effect, a separation distance between the light source 130 and the substrate 650 may be larger than a leakage length of the evanescent wave that leaks to the space between the light source 130 and the substrate 650 during propagation of the light from the light source 130 within the optical waveguide 140, and may be 2 μm or more, for example.

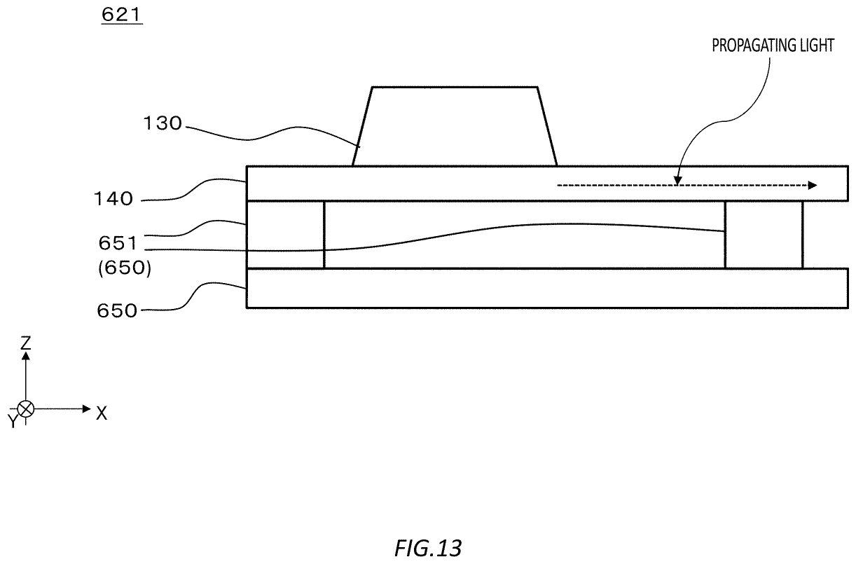

FIG. 13 is a schematic side view of an optical part 621 according to an implementation. The optical concentration measuring device 600 may include, instead of the optical part 620, an optical part 621 in which the light source 130 is formed on the positive side of the Z axis of the optical waveguide 140.

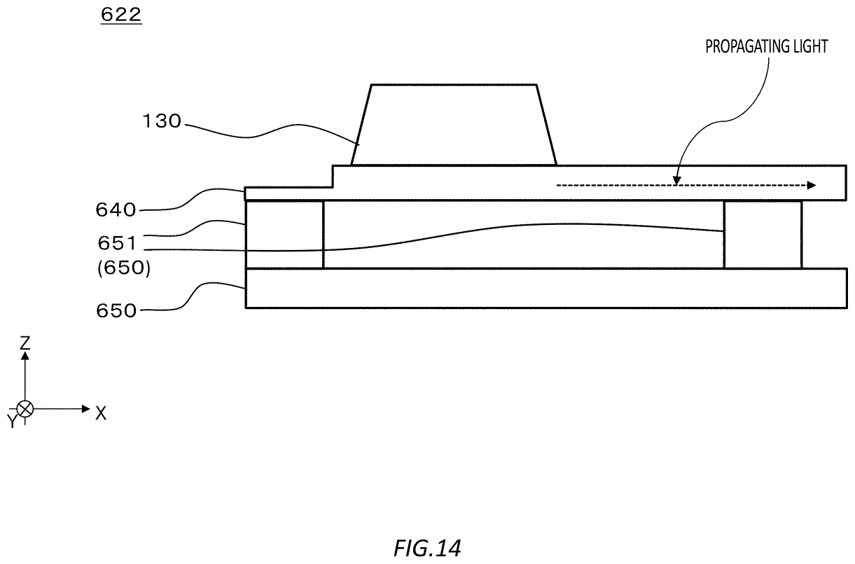

FIG. 14 is a schematic side view of an optical part 622 according to an implementation. The optical concentration measuring device 600 may include, instead of the optical part 320, an optical part 622 in which the light source 130 is formed on the positive side of the Z axis of an optical waveguide 640 and in which a film thickness of the optical waveguide 640 located on the positive side of the Z axis of the supporting portion 651 to be closest to the light source 130 is relatively thin. As shown in FIG. 14, the optical waveguide 640 may be provided with a step such that only said location has a thin film thickness, and the film thickness may become thicker from said location toward the positive side of the x axis in a multi-step or smooth manner. By including the optical part 622 having the optical waveguide 640 with a thin film thickness at said location, the optical concentration measuring device 600 can suppress the light from leaking to the substrate 650 via the supporting portion 651.

Note that, in a plurality of examples hereinafter, as an example, description is made with a space being provided between the light source 130 and the substrate 650, and at least a portion of the optical waveguide 140 being supported by the supporting portion 651 or the like of the substrate 650 or the like such that the entire surface thereof is exposed without being in contact with the substrate 650 or the like, similarly to the example of FIG. 12.

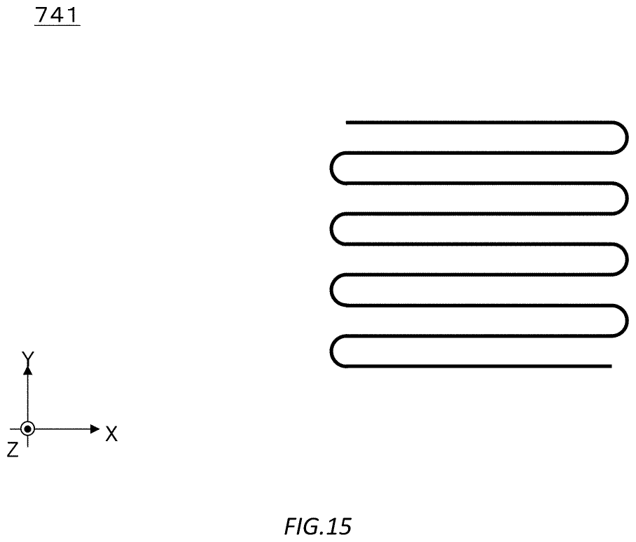

FIG. 15 is a schematic plan view of an optical waveguide 741 according to an implementation. In FIG. 1 to FIG. 14, the shape of the optical waveguide 140 or the like in the plan view has been illustrated in a simple way merely for the purpose of clarifying the description. The optical part 120 or the like of the optical concentration measuring device 100 may have, instead of the optical waveguide 140, an optical waveguide 741. The optical waveguide 741 has a zigzag shape in the plan view.

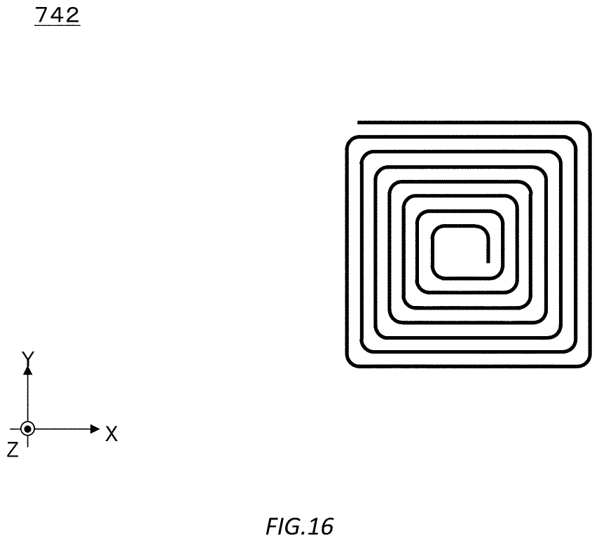

FIG. 16 is a schematic plan view of an optical waveguide 742 according to an implementation. The optical part 120 or the like of the optical concentration measuring device 100 or the like may have, instead of the optical waveguide 140, an optical waveguide 742. The optical waveguide 742 has a shape in which straight lines and curves are alternately and repeatedly circulating in the plan view.

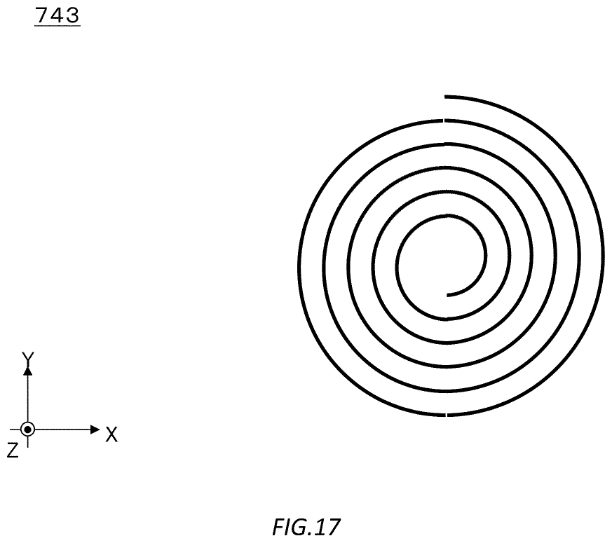

FIG. 17 is a schematic plan view of an optical waveguide 743 according to an implementation. The optical part 120 or the like of the optical concentration measuring device 100 or the like may have, instead of the optical waveguide 140, an optical waveguide 743. The optical waveguide 743 has a shape in which a curve is circulating in the plan view.

The optical waveguides 741, 742, 743 shown in FIG. 15 to FIG. 17 may be defined as including, at least in part, a bending portion that bends the propagation direction of the light propagating within the optical waveguide 741. In addition, at least a portion of the optical waveguide 741 or the like may also be defined as not being a straight line in the plan view. By including the optical part 120 or the like having the optical waveguide 741 or the like, the optical concentration measuring device 100 or the like can extend the optical path length per unit area on the surface of the substrate 150 or the like.

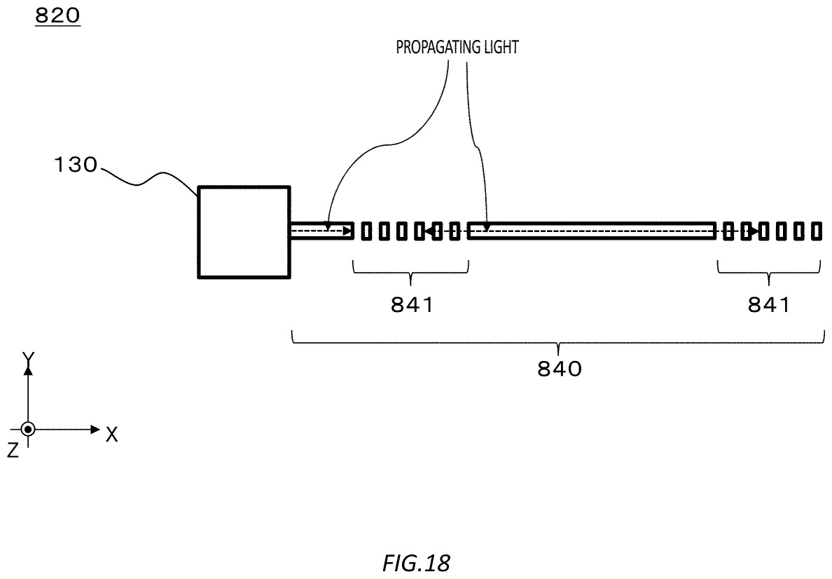

FIG. 18 is a schematic plan view of an optical waveguide 840 of an optical part 820 and a light source 130 according to an implementation. In FIG. 18, an illustration of other members included in the optical part 820 is omitted merely for the purpose of clarifying the invention. Also in the figures hereinafter, an illustration of some members is omitted for similar purposes, and duplicate descriptions will be omitted thereafter.

The optical concentration measuring device 100 or the like, may include, instead of the optical part 120 or the like, an optical part 820. In the optical part 820, the optical waveguide 840 includes one or more pairs of reflecting means 841 that, at least in part, reverse and thereafter restore the propagation direction of the light propagating within the optical waveguide 840. The reflecting means 841 may use reflection between a grating, a metal, a material having a high refractive index and a material having a low refractive index, for example. The optical waveguide 840 may also be defined as having, in at least a portion thereof, a section that is sandwiched by mirrors. By including the optical part 820, the optical concentration measuring device 100 or the like can extend the optical path length per unit area on the surface of the substrate 150 or the like.

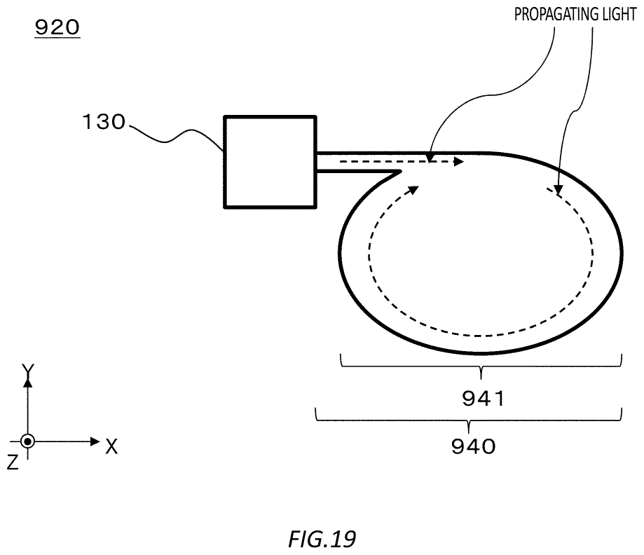

FIG. 19 is a schematic plan view of an optical part 920 according to an implementation. The optical concentration measuring device 100 or the like may include, instead of the optical part 120 or the like, an optical part 920. In the optical part 920, the optical waveguide 940 includes, in part, an elliptic circulating portion 941 that causes the light propagating within the optical waveguide 940 to circulate in an elliptical manner. A linear portion of the optical waveguide 940 other than the elliptic circulating portion 941 is referred to as a linear waveguide.

By including the optical part 920, the optical concentration measuring device 100 or the like can extend the optical path length per unit area on the surface of the substrate 150 or the like. In addition, when the linear waveguide is connected thereto for inputting light to the elliptic circulating portion 941, as in the implementation of FIG. 19, the optical concentration measuring device 100 or the like can control one or more modes of the input light with the waveguide width, and can cause the light to efficiently circulate in the elliptic circulating portion 941. In addition, when the linear waveguide that inputs light to the elliptic circulating portion 941 is connected at an angle along a tangent of the ellipse, the optical concentration measuring device 100 or the like can cause the light to efficiently circulate in the elliptic circulating portion 941.

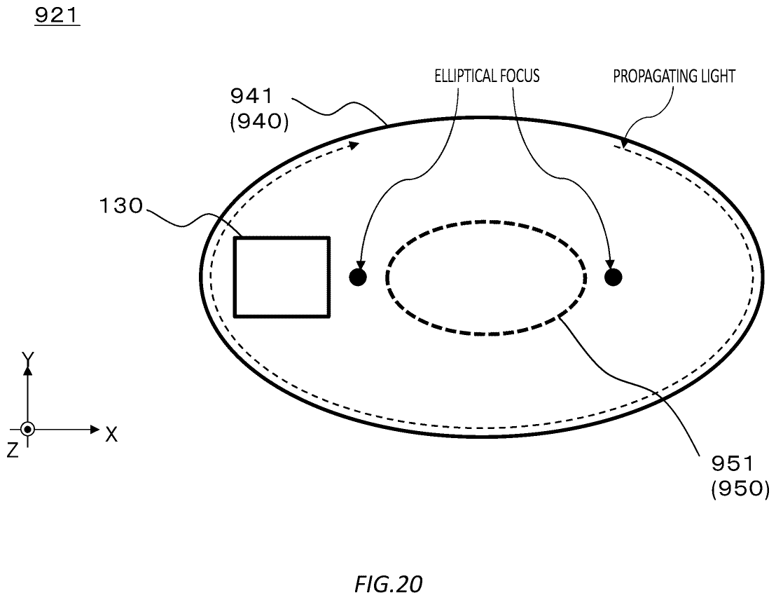

FIG. 20 is a schematic plan view of an optical part 921 according to an implementation.

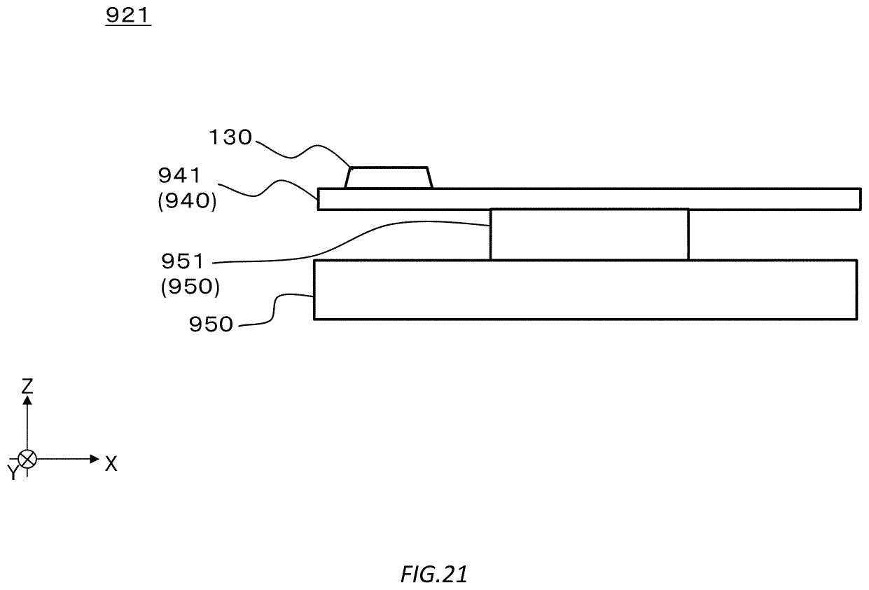

FIG. 21 is a schematic side view of the optical part 921 shown in FIG. 20. In FIG. 20, a supporting portion 951 of the substrate 950 of the optical part 921 is shown with a dash. The optical concentration measuring device 100 or the like may include, instead of the optical part 920, the optical part 921.

In the optical part 921, the optical waveguide 940 generally includes the elliptic circulating portion 941 that causes the light propagating within the optical waveguide 940 to circulate in an elliptical manner, that is, the optical waveguide 940 is an elliptic waveguide. The light source 130 is installed at a location that overlaps with the elliptic circulating portion 941 in the plan view. As in the implementation illustrated in FIGS. 20 and 21, when the light source 130 is installed within the elliptic circulating portion 941, the optical concentration measuring device 100 or the like can input the light emitted from the light source 130 more efficiently into the elliptic circulating portion 941. In addition, in this case, as shown in FIG. 20, the optical concentration measuring device 100 or the like can cause the light to circulate more efficiently in the elliptic circulating portion 941 by installing the light source 130 outside the elliptical focus of the elliptic circulating portion 941.

In addition, in the optical part 921, the supporting portion 951 of the substrate 950 supports the inside of the elliptical focus of the elliptic circulating portion 941 in the plan view, thereby allowing the entire surface of the outside of the elliptical focus of the elliptic circulating portion 941 to be exposed to a space within the acoustic cavity 110 without being in contact with the substrate 950. In this manner, the optical concentration measuring device 100 or the like can enhance the propagation efficiency of the light circulating on the outside of the elliptical focus of the elliptic circulating portion 941, and can increase contact between the evanescent wave that leaks to the outside of the optical waveguide 940 during the propagation of the light from the light source 130 within the optical waveguide 940 and the target component included in a gas that is present around the optical waveguide 940 and having a lower refractive index than the optical waveguide 940. In addition, the optical concentration measuring device 100 or the like can stabilize the support of the optical waveguide 940 by supporting, by the supporting portion 951 of the substrate 950, at least a portion of the region on the inside relative to the elliptical focus of the elliptic circulating portion 941.

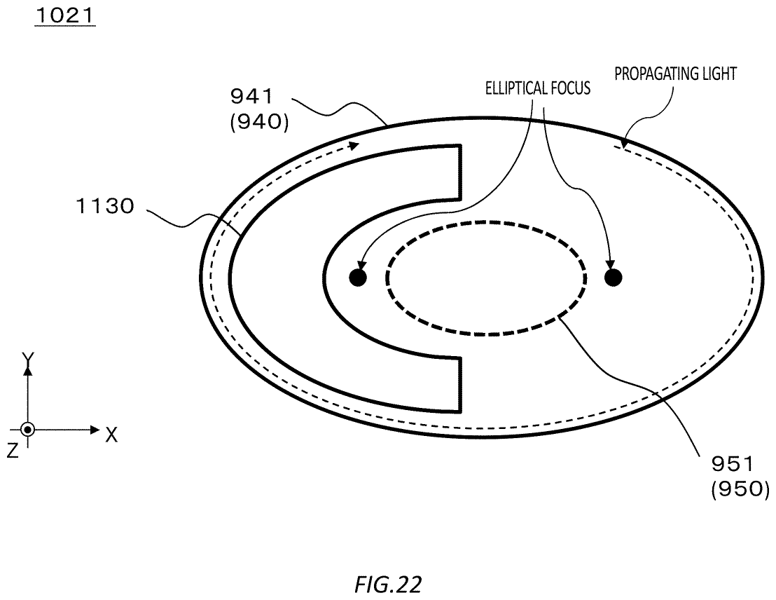

FIG. 22 is a schematic plan view of an optical part 1021 according to an implementation. In FIG. 22, the supporting portion 951 of the substrate 950 of the optical part 1021 is shown with a dashed line. The optical concentration measuring device 100 or the like may include, instead of the optical part 921, the optical part 1021 having a light source 1130 that is configured such that a portion of the contour thereof is generally along the contour of the elliptic circulating portion 941. As in the implementation of FIG. 22, the optical concentration measuring device 100 or the like can increase the optical power to be input to the elliptic circulating portion 941 by installing the light source 1130 on the outside of the elliptical focus of the elliptic circulating portion 941 and increasing the surface area of the light source 1130.

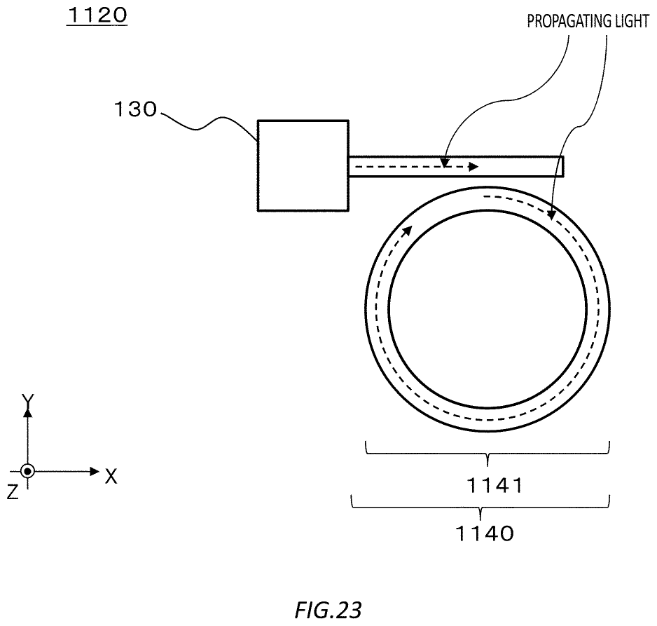

FIG. 23 is a schematic plan view of an optical waveguide 1140 of an optical part 1120 and the light source 130 according to an implementation. The optical concentration measuring device 100 or the like, may include, instead of the optical part 120 or the like, an optical part 1120. In the optical part 1120, the optical waveguide 1140 includes a ring-shaped circulating portion 1141 that causes, at least in part, the light propagating within the optical waveguide 1140 to circulate in an elliptical or circular manner. A linear portion, other than the ring-shaped circulating portion 1141, of the optical waveguide 1140 is referred to as the linear waveguide. In the optical waveguide 1140, the connecting portion between the ring-shaped circulating portion 1141 and the linear waveguide is a directional coupler, and the separation distance between the ring-shaped circulating portion 1141 and the linear waveguide at said connecting portion is adjustable.

By including the optical part 1120, the optical concentration measuring device 100 or the like can extend the optical path length per unit area on the surface of the substrate 150 or the like. In addition, the optical concentration measuring device 100 or the like can efficiently introduce light into the ring-shaped circulating portion 1141 by controlling the distance between the linear waveguide and the ring-shaped circulating portion 1141, as in the implementation of FIG. 23.

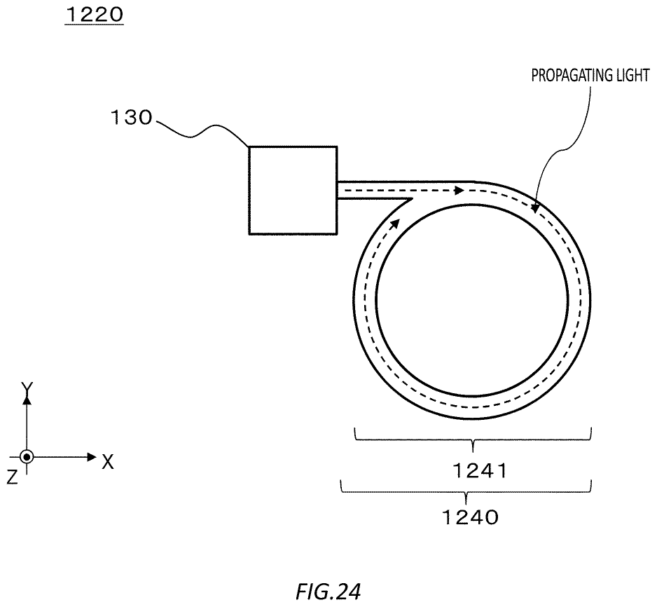

FIG. 24 is a schematic plan view of an optical waveguide 1240 of an optical part 1220 and the light source 130 according to an implementation. The optical concentration measuring device 100 or the like may include, instead of the optical part 1120, an optical part 1220. In the optical part 1220, there is not gap between the ring-shaped circulating portion 1241 and the linear waveguide, that is, they are integrally formed. By including the optical part 1220, the optical concentration measuring device 100 or the like can eliminate the need of fine-tuning of dimensions that is less than the waveguide width and can be easily manufactured.

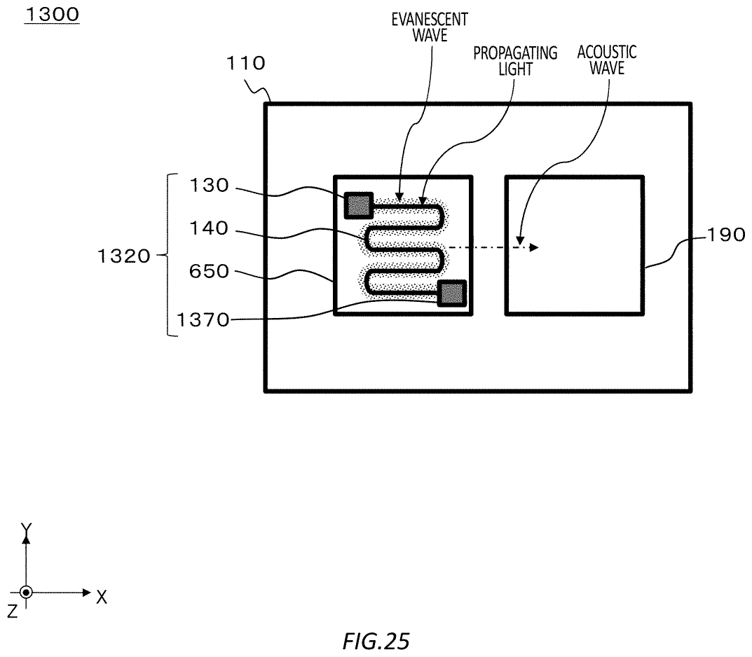

FIG. 25 is a schematic plan view of an optical concentration measuring device 1300 according to an implementation.

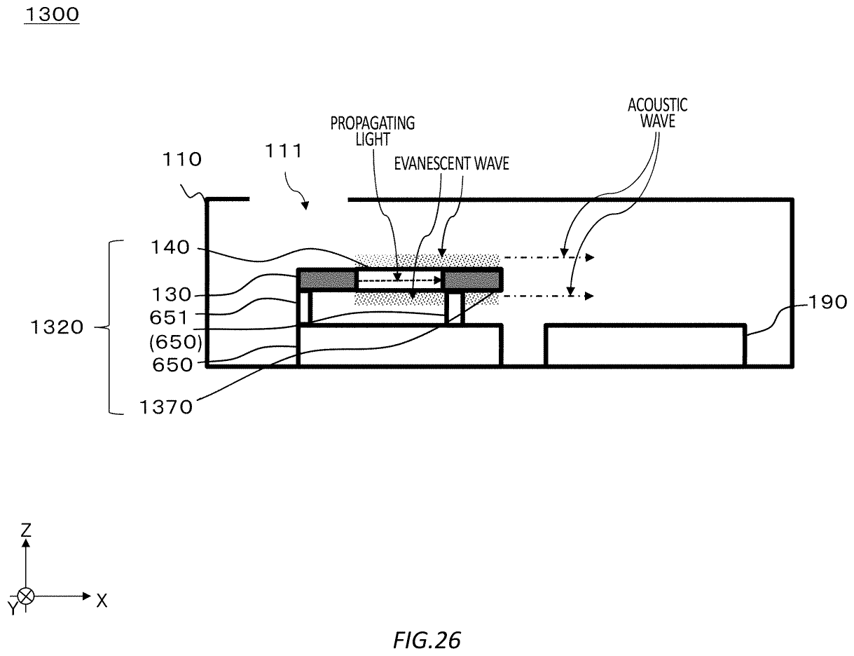

FIG. 26 is a schematic side view of the optical concentration measuring device 1300 shown in FIG. 25. As a difference from the optical concentration measuring device 100, the optical concentration measuring device 1300 includes, instead of the optical part 120, an optical part 1320 additionally having an optical receiver 1370.

The optical receiver 1370 detects the light from the light source 130. In the present example, the optical receiver 1370 is provided to be in direct contact with an end that is opposite to the end with which the light source 130 is in direct contact, among both ends of the optical waveguide 140. In addition, the light source 130 may adjust the output of the light to be emitted, according to the detection result of the optical receiver 1370. By including such an optical part 1320, the optical concentration measuring device 1300 can detect the light output of the light source 130 and perform signal processing according to said light output, such as adjusting the light output of the light source 130, for example. In the present example, in addition, the optical receiver 1370 is installed on one substrate 650 together with another member such as the light source 130. In this manner, the optical concentration measuring device 1300 can be made more compact.

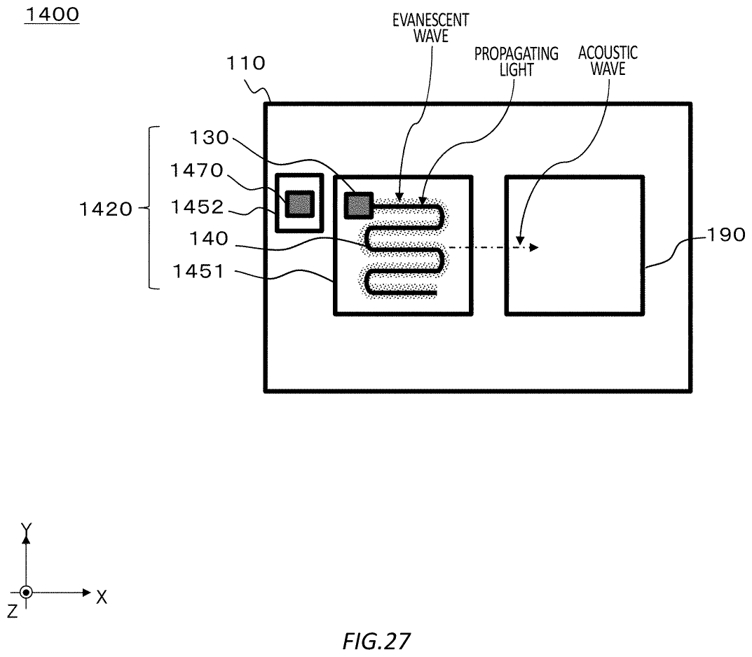

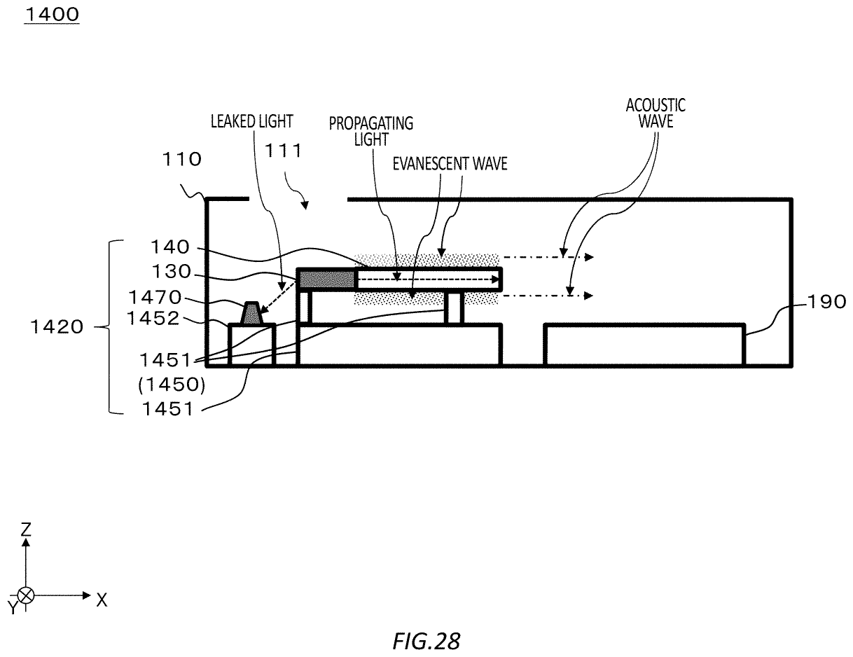

FIG. 27 is a schematic plan view of an optical concentration measuring device 1400 according to an implementation. FIG. 28 is a schematic side view of the optical concentration measuring device 1400 shown in FIG. 27. As a difference from the optical concentration measuring device 1300, the optical concentration measuring device 1400 includes, instead of the optical part 1320, an optical part 1420. In the optical part 1420, the optical receiver 1470 is provided on another substrate 1452 other than the substrate 1450 on which another member such as the light source 130 is provided. In addition, the optical receiver 1470 is provided next to the light source 130, and detects the leaked light, shown with a dashed line in FIG. 28, from the light source 130 located on the supporting portion 1451 of the substrate 1450. By including 1420 such an optical part, the optical concentration measuring device 1400 can design only the optical receiver 1470 differently from another member such as the light source 130.

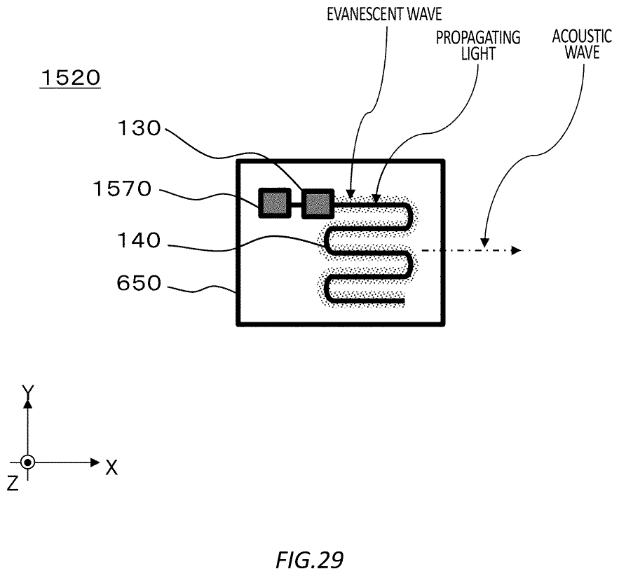

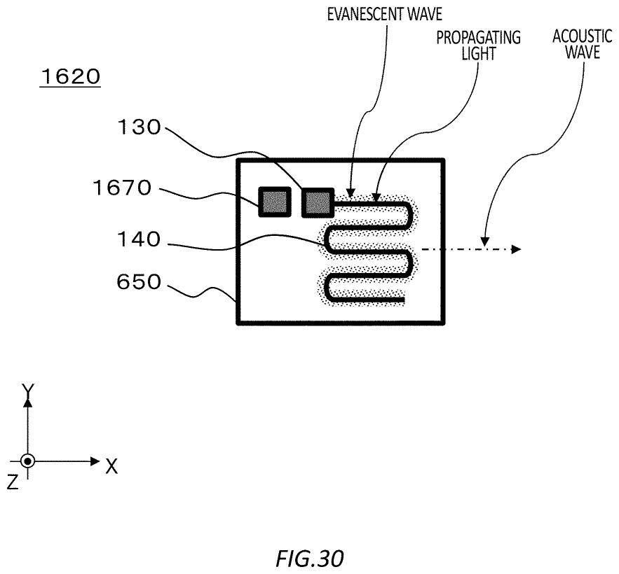

FIG. 29 is a schematic plan view of an optical part 1520 according to an implementation. FIG. 30 is a schematic plan view of an optical part 1620 according to an implementation. The optical concentration measuring device 1300 may include, instead of the optical part 1320, optical parts 1520, 1620 having optical receivers 1570, 1670 provided next to the light source 130. In the optical part 1520 in the example of FIG. 29, the optical receiver 1570 is connected to the light source 130 via a waveguide. On the other hand, in the optical part 1620 in the example of FIG. 30, the optical receiver 1670 is not connected to the light source 130.

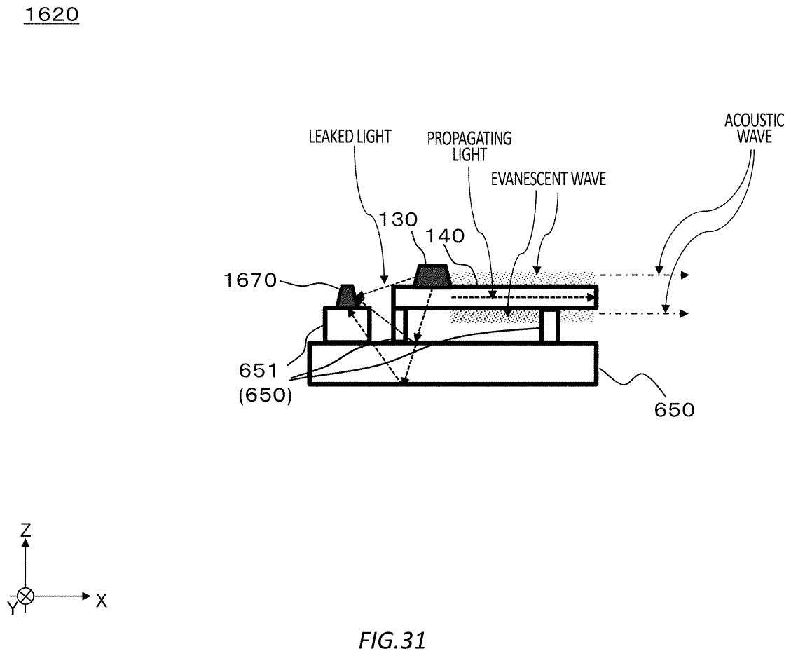

FIG. 31 is a schematic side view of the optical part 1620 shown in FIG. 30. The optical receiver 1670 is provided next to the light source 130 on one substrate 650, and detects the leaked light, illustrated with a dashed line in FIG. 31, from the light source 130 located on the supporting portion 651 of the substrate 650. As shown in FIG. 31, the leaked light detected by the optical receiver 1670 may include direct light that directly reaches the optical receiver 1670 from the light source 130 and reflected light that reaches thereto after being reflected on the surface or back surface of the substrate 650. By including such an optical part 1620, the optical concentration measuring device 1300 can detect, among the light from the light source 130, the light output of the light source 130 without decreasing the optical power used to measure the concentration of the target component in the gas.

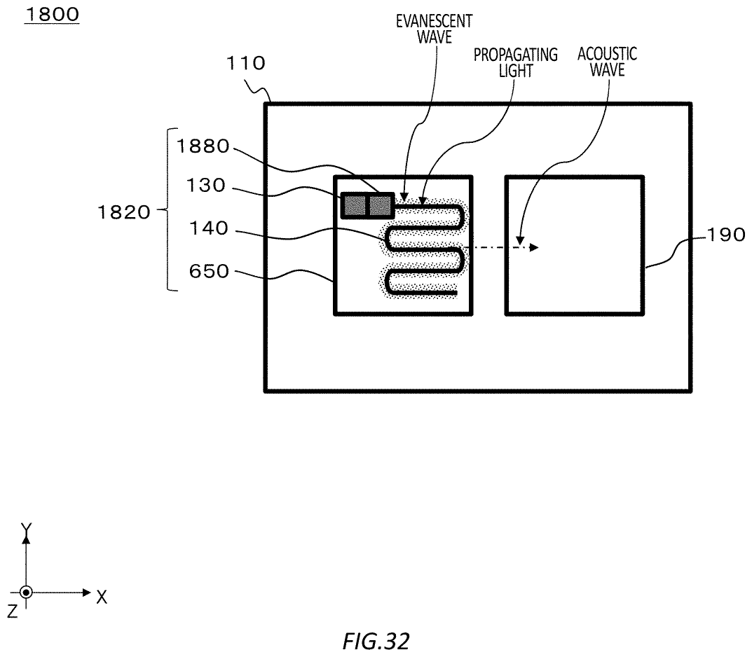

FIG. 32 is a schematic plan view of an optical concentration measuring device 1800 according to an implementation. As a difference from the optical concentration measuring device 100, the optical concentration measuring device 1800 includes, instead of the optical part 120, an optical part 1820 that additionally has an optical filter. 1880 arranged between the light source 130 and the optical waveguide 140.

Among the light from the light source 130, the optical filter 1880 transmits a wavelength that is absorbed by the target component in the gas, and shields wavelengths other than said wavelength. By including such an optical part 1820, the optical concentration measuring device 1800 can prevent components other than the target component in the gas from absorbing the light from the light source 130 to generate acoustic waves, thereby enhancing the measurement precision of the concentration of the target component.

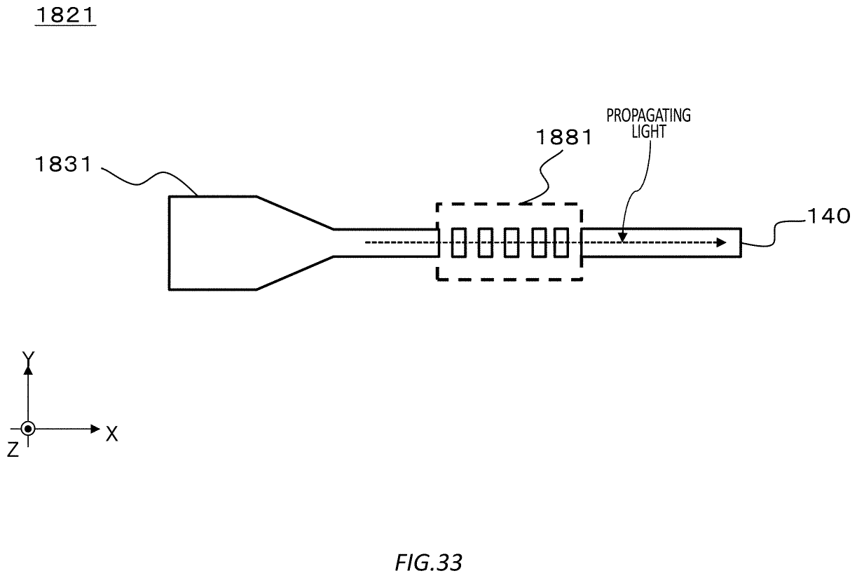

FIG. 33 is a schematic plan view of an optical filter. 1881, a light source 1831, and an optical waveguide 140 of an optical part 1821 according to an implementation. The optical concentration measuring device 1800 may include, instead of the optical part 1820, an optical part 1821 having an optical filter 1881 consisting of a grating surrounded by a dashed frame in FIG. 33 and a light source 1831 optically connected to said optical filter 1881.

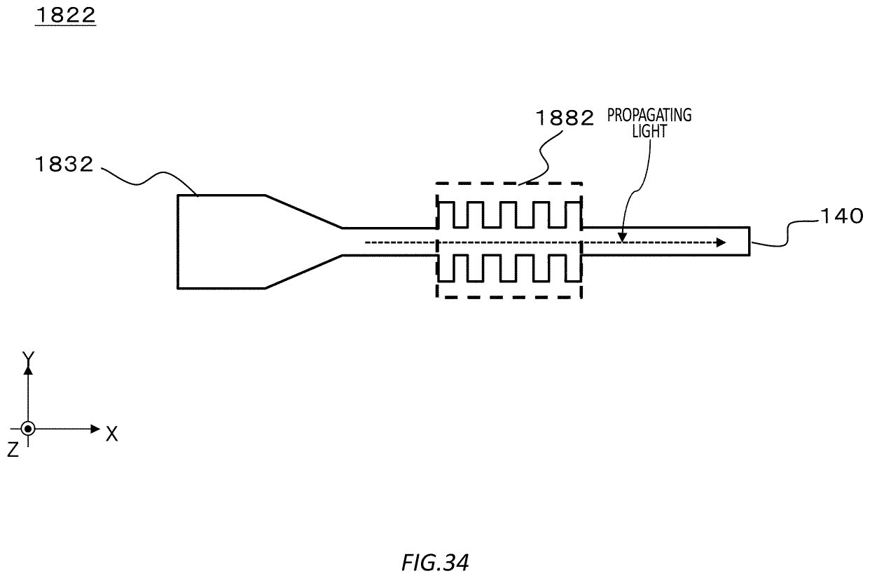

FIG. 34 is a schematic plan view of an optical filter. 1882, a light source 1832, and an optical waveguide 140 of an optical part 1822 according to an implementation. The optical concentration measuring device 1800 may include, instead of the optical part 1820, an optical part 1822 having an optical filter 1882 consisting of a side grating surrounded by a dashed frame in FIG. 34 and a light source 1832 optically connected to said optical filter 1882.

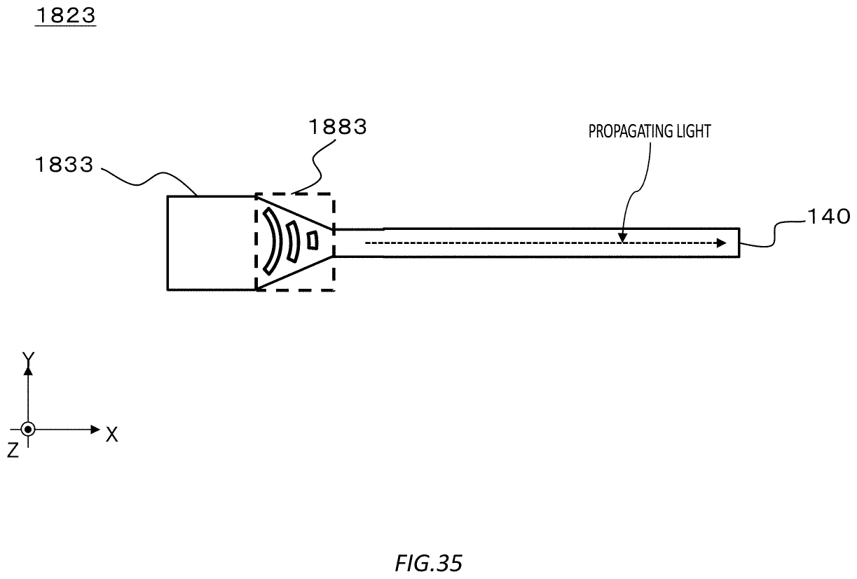

FIG. 35 is a schematic plan view of an optical filter. 1883, a light source 1833, and an optical waveguide 140 of an optical part 1823 according to an implementation. The optical concentration measuring device 1800 may include, instead of the optical part 1820, an optical part 1823 having an optical filter 1883 consisting of a focusing grating surrounded by a dashed frame in FIG. 35 and a light source 1833 optically connected to said optical filter 1883.

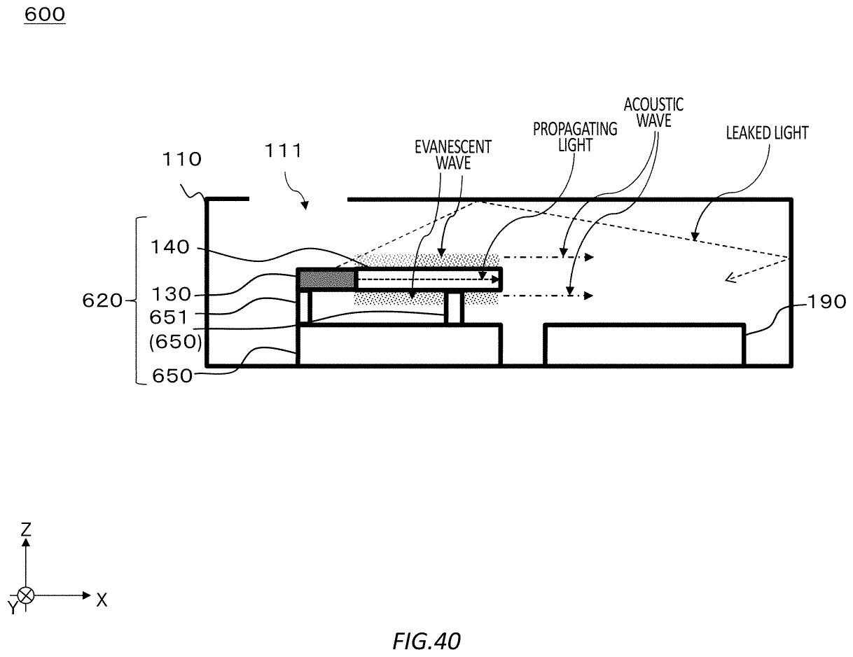

Note that, in each of the optical part 1821 or the like illustrated in FIG. 33 to FIG. 35, the wavelengths that are not selected by the grating may be emitted into the acoustic cavity 110. Note that, for example, by designing the reflection optical path length within the acoustic cavity 110 to be sufficiently shorter than the optical path length of the optical waveguide 140, components other than the target component in the gas can absorb the light emitted into the acoustic cavity 110 to suppress acoustic waves from being generated, thereby enhancing the measurement precision of the concentration of the target component. This will be described below with reference to FIG. 40.

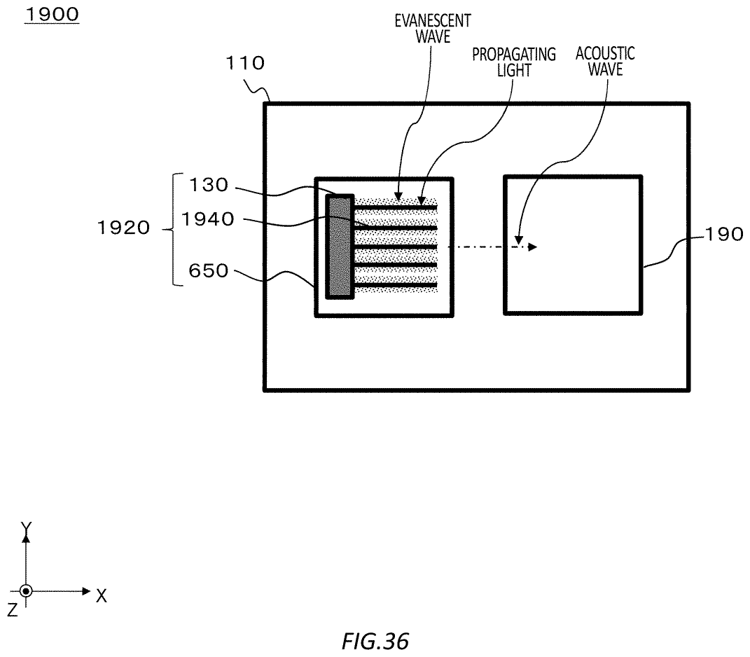

FIG. 36 is a schematic plan view of an optical concentration measuring device 1900 according to an implementation. As a difference from the optical concentration measuring device 100, the optical concentration measuring device 1900 includes, instead of the optical part 120, an optical part 1920 having a plurality of optical waveguides 1940 that are optically connected to one light source 130 on one substrate 650. As described above, a sum of the total length of the plurality of optical waveguides 1940 is, for example, approximately 2 cm.

Although, when the light propagates through the waveguide for a long distance, the light is attenuated due to propagation loss, according to the optical concentration measuring device 1900, by including the optical part 1920 in which a plurality of optical waveguides 1940 each being short is connected to the light source 130, such propagation loss can be reduced while securing the sum of the optical path lengths.

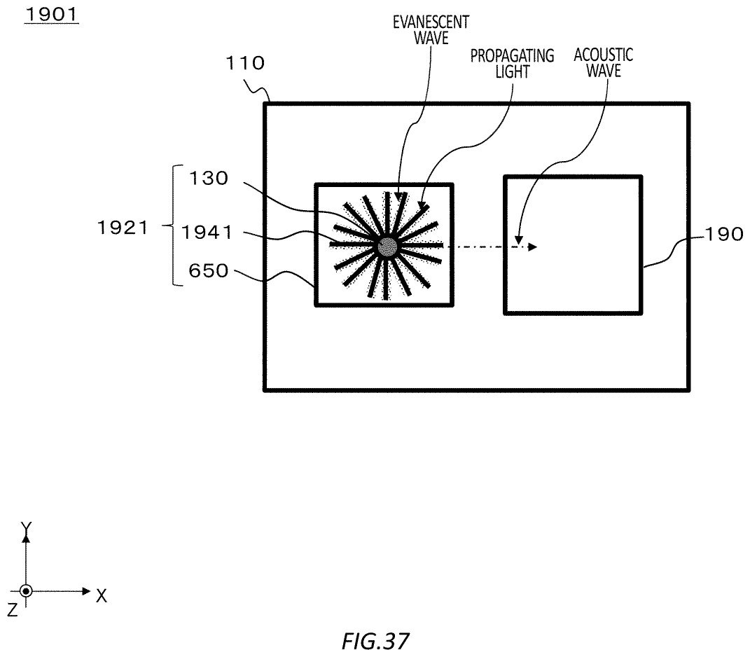

FIG. 37 is a schematic plan view of an optical concentration measuring device 1901 according to an implementation. As a difference from the optical concentration measuring device 1900, the optical concentration measuring device 1901 includes, instead of the optical part 1920, an optical part 1921 in which a plurality of optical waveguides 1941 optically connected to one light source 130 is arranged to radially extend from the light source 130. According to the optical concentration measuring device 1901 including such an optical part 1921, the propagation loss can also be reduced while securing the sum of the optical path lengths.

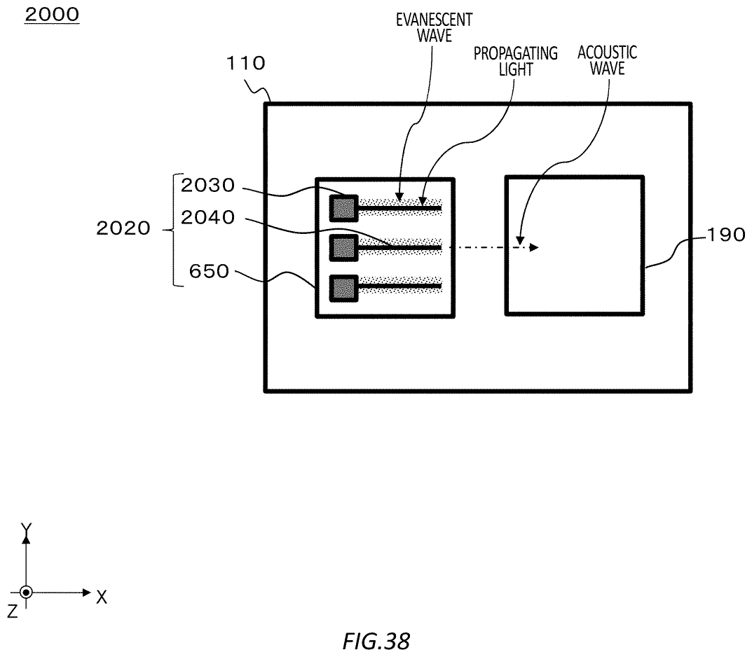

FIG. 38 is a schematic plan view of an optical concentration measuring device 2000 according to an implementation. As a difference from the optical concentration measuring device 100, the optical concentration measuring device 2000 includes, instead of the optical part 120, an optical part 2020 having a plurality of light sources 2030 that are optically connected to a plurality of optical waveguides 2040 on one substrate 650.

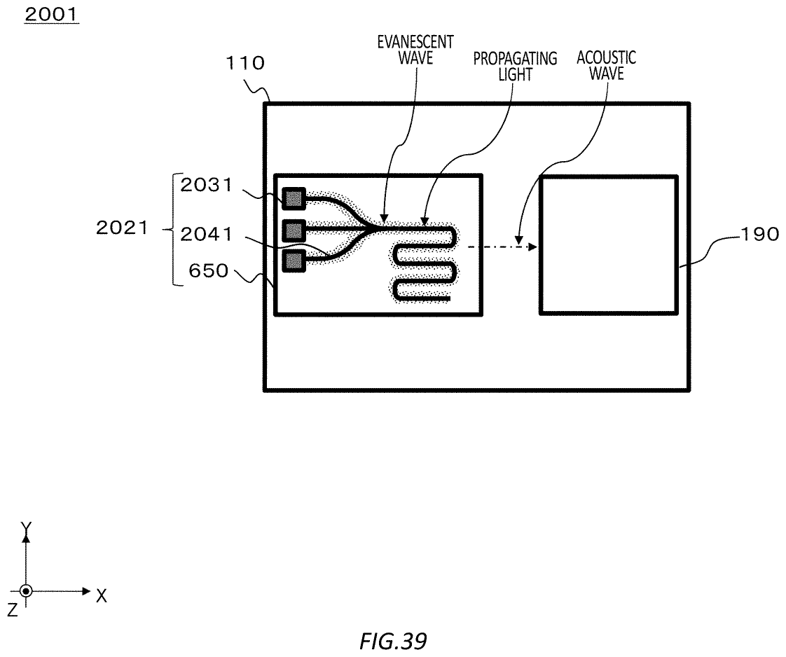

FIG. 39 is a schematic plan view of an optical concentration measuring device 2001 according to an implementation. As a difference from the optical concentration measuring device 2000, the optical concentration measuring device 2001 includes, instead of the optical part 2020, an optical part 2021 having a plurality of light sources 2031 that are optically connected to one optical waveguide 2041 on one substrate 650.

When many light sources are used, the light emission power is increased, which increases the photoacoustic signal and improves gas sensitivity. Instead, the electrical power consumption for driving the light source is large. On the other hand, when less light sources are used, the light emission power is decreased, which reduces the photoacoustic signal and lowers the gas sensitivity. Instead, the electrical power consumption for driving the light source is decreased. According to the optical concentration measuring devices 2000, 2001 illustrated in FIG. 38 and FIG. 39, by including the optical parts 2020, 2021 described above, the optical power and the electrical power consumption can be adjusted by selectively driving one or more of the plurality of light sources 2030, 2031.

FIG. 40 is a schematic side view of the optical concentration measuring device 600 shown in FIG. 6. In FIG. 40, unlike in FIG. 6, the leaked light from the light source 130 is illustrated with a dashed line. In the optical concentration measuring device 600 or the like, an average optical path length of the light, among the light from the light source 130, that is emitted, without entering the optical waveguide 140, to a space within the acoustic cavity 110 and reflected by the inner wall of the acoustic cavity 110 may be shorter than an effective optical path length of the optical waveguide 140. By being thus designed, the optical concentration measuring device 600 or the like can suppress the leaked light that is emitted into the space in the acoustic cavity 110 and reflected by the inner wall from being absorbed by components other than the target component in the gas and generating acoustic waves, thereby enhancing the measurement precision of the concentration of the target component.

In order to make the average optical path length of the leaked light shorter than the optical path length of the optical waveguide 140, the average reflectance of the inner wall of the acoustic cavity 110 may be set to be 70% or less, for example. Specifically, when the average reflectance of the inner wall of the acoustic cavity 110 is defined as R and the leaked light is reflected N times by the inner wall, an intensity of the leaked light becomes R\hat{ }N. The number of times of reflection by the inner wall required to make the intensity of the leaked light equal to or less than 1/10, for example, is twenty-two times when R=0.9 and eleven times when R=0.8. The number of times of reflection by the inner wall required to make the intensity of the leaked light equal to or less than 1/10 is seven times when R=0.7, five times when R=0.6, and four times when R=0.5. Therefore, in the optical concentration measuring device 600, by making the average reflectance of the inner wall 70% or less, the average optical path length of the leaked light can be made ⅓ or less compared to when the average reflectance is 90%. Thus, it is understood that, when the average reflectance of the inner wall is made 70% or less, the effect of reduction in the optical path length of the leaked light relative to the reduction of the reflectance is large.

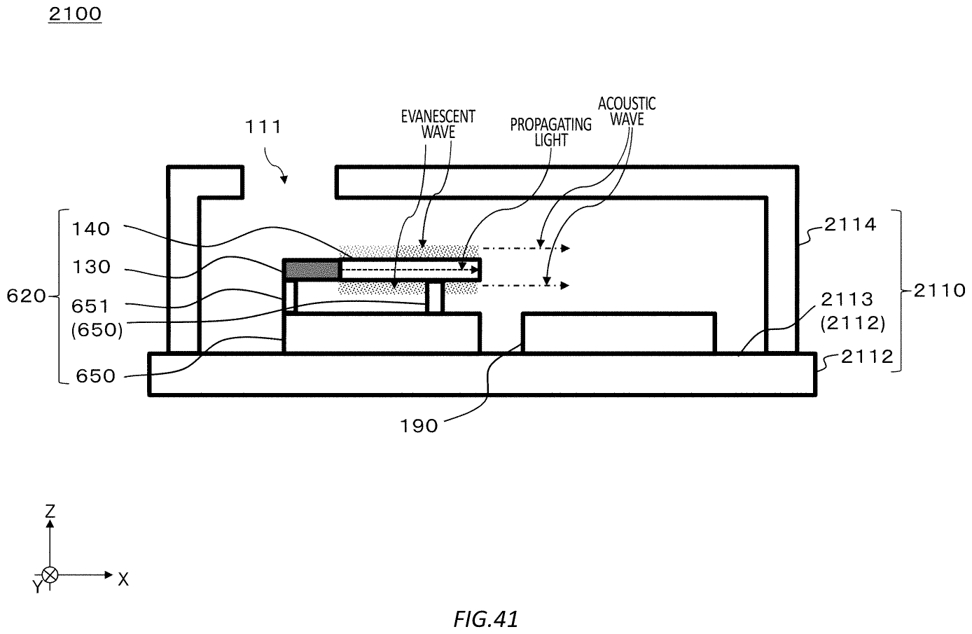

FIG. 41 is a schematic side view of an optical concentration measuring device 2100 according to an implementation. As a difference from the optical concentration measuring device 600, the optical concentration measuring device 2000 includes, instead of the acoustic cavity 110, an acoustic cavity 2110 having a foundation portion 2112 and a lid portion 2114, which are separate from each other.

In the acoustic cavity 2110, the foundation portion 2112 is a portion on which the optical part 120 and the acoustic transducer 190 are installed on the surface 2113, and the lid portion 2114 is a portion in which the gas inlet opening 111 is formed and to which the foundation portion 2112 is attached. By including such an acoustic cavity 2110, the optical concentration measuring device 2100 enables to be easily manufactured by installing members to be installed in the acoustic cavity 2110, for example, the optical part 120 and the acoustic transducer 190 on the surface 2113 of the foundation portion 2112, and thereafter attaching the lid portion 2114 to the foundation portion 2112.

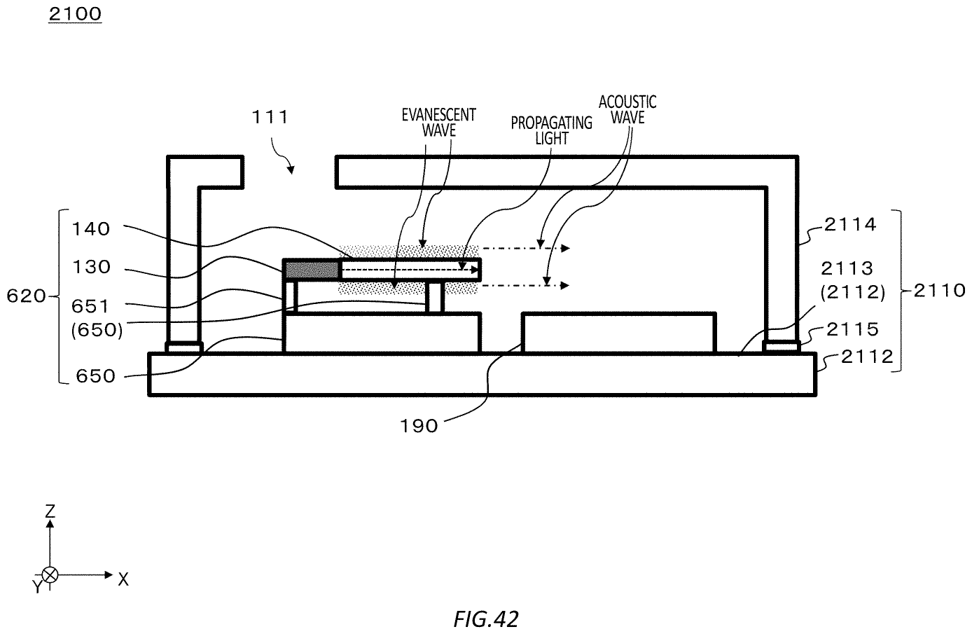

FIG. 42 is a schematic side view of an optical concentration measuring device 2100 according to an implementation. As a difference from the optical concentration measuring device 2100 shown in FIG. 41, the optical concentration measuring device 2100 shown in FIG. 42 has the lid portion 2114 adhered to the foundation portion 2112 by an adhesive 2115. In this manner, an internal space of the acoustic cavity 2110 communicates to the external space of the acoustic cavity 2110 only via the gas inlet opening 111. According to the optical concentration measuring device 2100 shown in FIG. 42, as compared to a case where the lid portion 2114 is mounted on the foundation portion 2112 without being adhering, the adhesion between the lid portion 2114 and the foundation portion 2112 is increased and the airtightness in the internal space of the acoustic cavity 2110 is increased, thereby preventing attenuation of the pressure fluctuation within the acoustic cavity 2110.

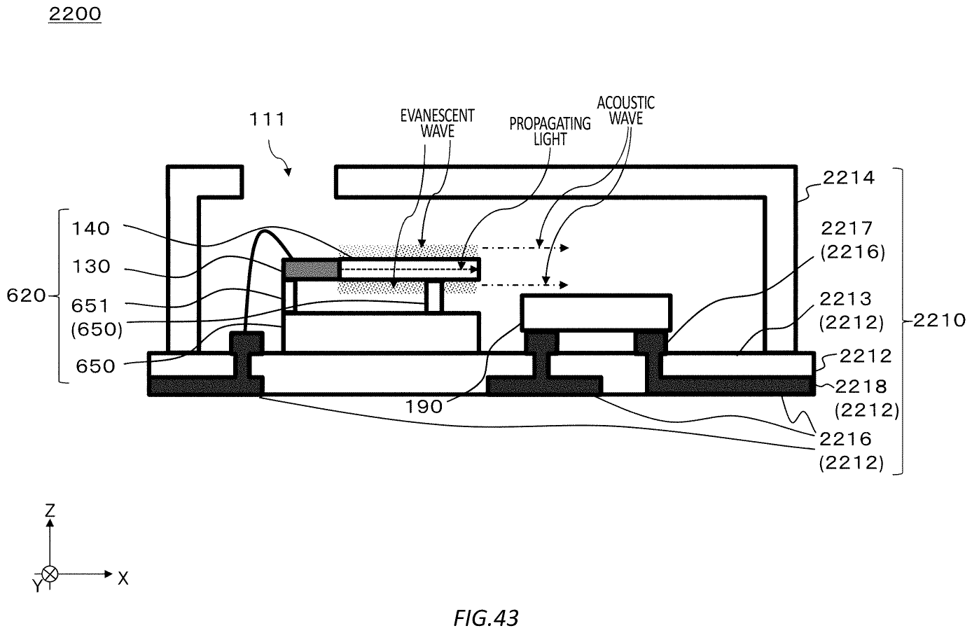

FIG. 43 is a schematic side view of an optical concentration measuring device 2200 according to an implementation. As a difference from the optical concentration measuring device 2100 shown in FIG. 41, in the optical concentration measuring device 2200, the foundation portion 2212 has a conductive portion 2216. The conductive portion 2216 includes an inner contact end 2217 formed on the surface 2213 of the foundation portion 2212 and an outer contact end 2218 exposed to the external space of the acoustic cavity 2210. The inner contact end 2217 is electrically connected to at least any of the light source 130 or the acoustic transducer 190. According to the optical concentration measuring device 2200, by including such an acoustic cavity 2210, communication can be performed between the inside and outside of the acoustic cavity 2210.

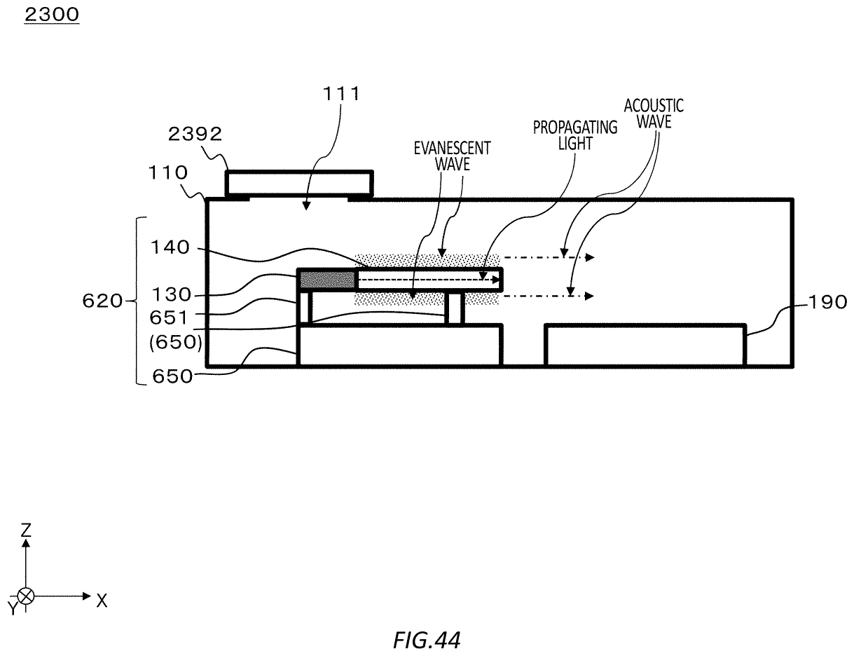

FIG. 44 is a schematic side view of an optical concentration measuring device 2300 according to an implementation. As a difference from the optical concentration measuring device 600, the optical concentration measuring device 2300 additionally includes a dustproof filter 2392. The dustproof filter 2392 is attached to the gas inlet opening 111 of the acoustic cavity 110 to seal the gas contained in the acoustic cavity 110. By including such a dustproof filter 2392, the optical concentration measuring device 2300 can prevent foreign substances from entering into the acoustic cavity 110.

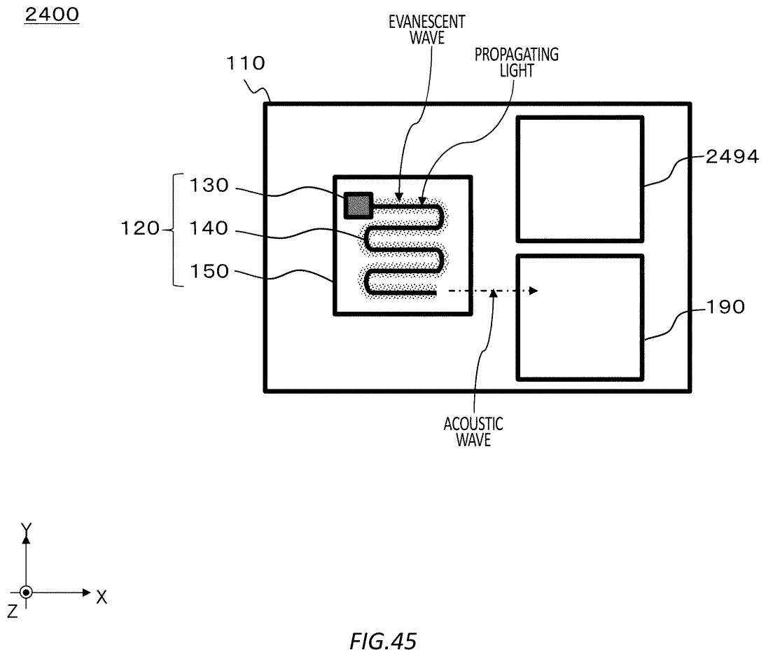

FIG. 45 is a schematic plan view of an optical concentration measuring device 2400 according to an implementation. As a difference from the optical concentration measuring device 100, the optical concentration measuring device 2400 additionally includes an electrical circuit 2494 installed within the acoustic cavity 110. The electrical circuit 2494 is electrically connected to at least any of the light source 130 or the acoustic transducer 190.

The electrical circuit 2494 may function as a circuit for driving the light source, for example, and may adjust the output of the light to be emitted from the light source 130. Additionally or alternatively, the electrical circuit 2494 may function as a concentration computing circuit, for example, and may compute the concentration of the target component in the gas based on the output by the acoustic transducer 190. In addition, the electrical circuit 2494 may additionally or alternatively function as an analog-digital conversion circuit, a digital data transmission circuit or the like.

In an acoustic cavity that utilizes reflection on the inside thereof such as the measurement device in the comparative example described above, since it is difficult to control the reflectance of the light on the circuit surface when the electrical circuit is installed within the acoustic cavity, the electrical circuit is installed outside the acoustic cavity. To the contrary, according to the optical concentration measuring device 2400, since the electrical circuit 2494 can be installed within the acoustic cavity 110 by separating the optical path from the acoustic cavity 110, the optical concentration measuring device 2400 can be made more compact, and integration of the optical concentration measuring device 2400 into another device can be facilitated.

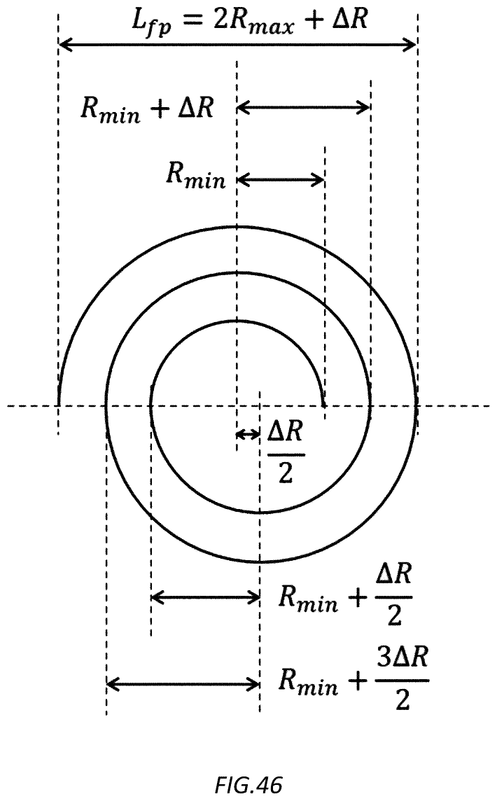

FIG. 46 illustrates an example of a method of calculating a total length of the optical waveguide 743 shown in FIG. 17, which has a circulating shape with a curve in a plan view. The total length Lₚᵣₒₚ of the optical waveguide 743 can be calculated as follows:

[Equation 1] ΔR is a sum of the waveguide width W_{waveguide} and the waveguide spacing W_{spacing}. Rmin is minimum radius of curvature, and n is the number of circulation. In addition, a footprint length Lfp, which is a length of one side of a square surrounding the optical waveguide 743 in the plan view, is obtained by addingΔR to twice the maximum radius of curvature Rmax.

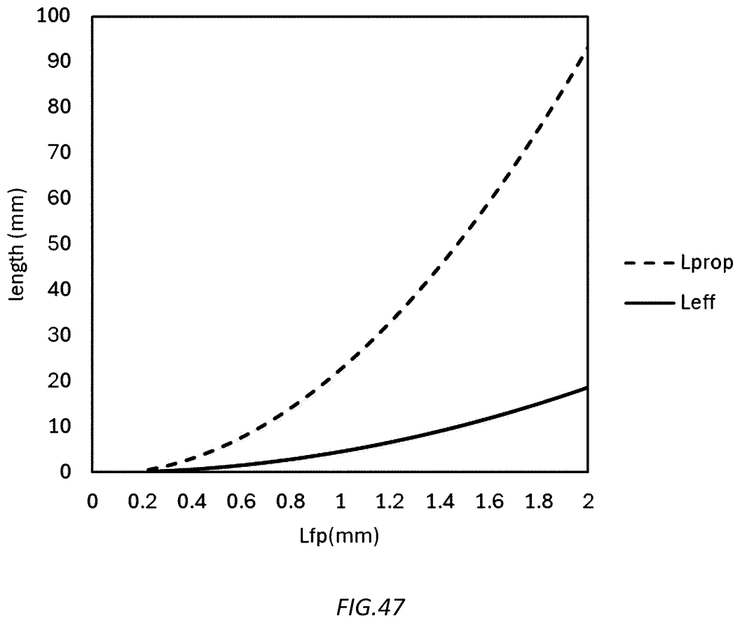

FIG. 47 is a graph representing a relationship between a length of one side of a square surrounding the optical waveguide 743 in the plan view and each of the total length and an effective optical path length of the optical waveguide 743. The horizontal axis of the graph indicates the footprint length [mm], and the vertical axis of the graph indicates an optical path length of the optical waveguide 743, that is, the total length [mm] and the effective optical path length [mm]. In FIG. 47, the curve illustrated with a dashed line on the top side represents the total length Lₚᵣₒₚ of the optical waveguide 743, and the curve illustrated on the bottom side represents ηLₚᵣₒₚ, which is obtained by multiplying the total length Lₚᵣₒₚ by a leakage efficiency η of the optical waveguide 743, that is, the effective optical path length. In the present example, η is 20%.

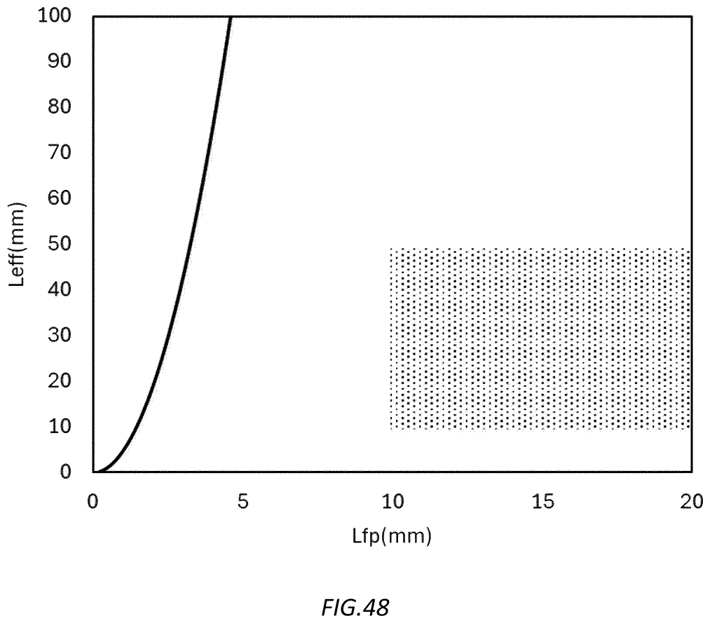

FIG. 48 is a graph representing a relationship between a length of one side of an occupied area of the square and the effective optical path length of the optical waveguide 743, according to the optical concentration measuring device 600 or the like including the optical waveguide 743. The horizontal axis of the graph indicates the footprint length Lfp [mm], and the vertical axis of the graph indicates the effective optical path length Leff [mm]. In FIG. 48, a dimensional range of the optical path of the measurement device in the comparative example described above is indicated by a dotted area. As shown in FIG. 48, in order to secure an effective optical path length Leff of 10 to 50 mm, a footprint length Lfp of 10 mm to 20 mm is required for the measurement device in the comparative example, whereas the optical concentration measuring device 600 or the like including the optical waveguide 743 only requires a footprint length Lfp of less than 5 mm.

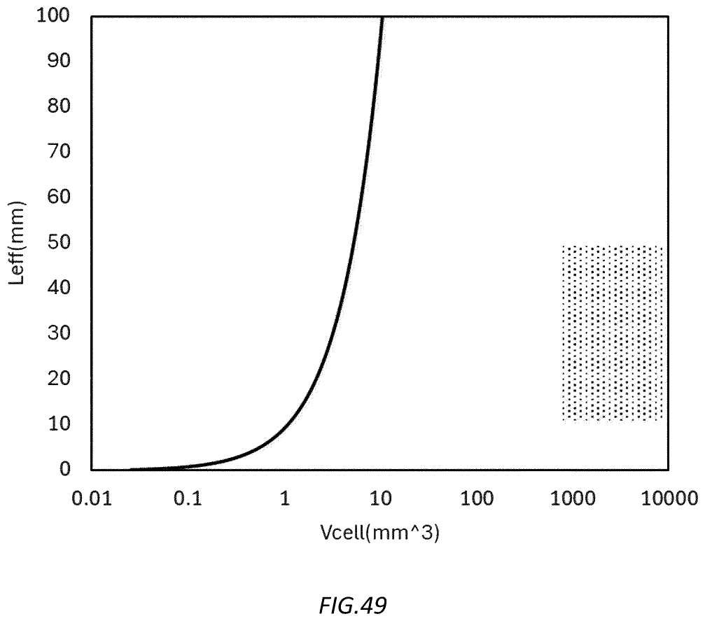

FIG. 49 is a graph representing a relationship between a volume of the optical concentration measuring device 600 or the like and the effective optical path length of the optical waveguide 743 in the example of FIG. 48. The horizontal axis of the graph indicates a volume Vcell [mm³] of the optical concentration measuring device 600 or the like, and the vertical axis of the graph indicates the effective optical path length Leff [mm]. The curve illustrated in FIG. 49 represents a cell volume Vcell of the optical concentration measuring device 600 or the like including the optical waveguide 743. In FIG. 49, a dimensional range of the measurement device in the comparative example is illustrated with a dotted area.

In the optical concentration measuring device 600 or the like including the optical waveguide 743, the height of the acoustic cavity 110, that is, the cell height of the optical concentration measuring device 600 or the like is assumed to be 0.5 mm, as an example. In this case, as shown in FIG. 49, in order to secure an effective optical path length Leff of 10 to 50 mm, while the measurement device of the comparative example described above requires a cell volume Vcell of 700 mm³ to 8000 mm³, the optical concentration measuring device 600 or the like including the optical waveguide 743 only requires a cell volume Vcell of less than 10 mm³. In this manner, according to the optical concentration measuring device 600 or the like, the size thereof can be made significantly more compact compared to the measurement device in the comparative example.

While the present invention has been described hereinabove by using the embodiment, a technical scope of the present invention is not limited to a scope of the above-described embodiment. It is apparent to persons skilled in the art that various changes or improvements may be made to the embodiment described above. It is also apparent from the scope of the claims that the embodiments added with such changes or improvements can be included in the technical scope of the invention.

It should be noted that each process of the operations, procedures, steps, stages, and the like performed by the device, system, program, and method shown in the claims, specification, and drawings may be executed in any order as long as the order is not particularly explicitly indicated by “before”, “prior to”, or the like and as long as an output from a previous process is not used in a later process. Even if the operation flow is described using phrases such as “first” or “next” in the claims, the description, and the drawings, it does not necessarily mean that it must be performed in this order.

### OTHER POSSIBLE CLAIMS

### Item 1

An optical concentration measuring device for measuring a concentration of a target component in a gas, the optical concentration measuring device comprising:
an acoustic cavity that has a gas inlet opening and contains the gas introduced therein via the gas inlet opening;
an optical part installed within the acoustic cavity; and
an acoustic transducer installed within the acoustic cavity,
wherein the optical part includes one or more of each of a light source that emits light including a wavelength that is absorbed by the target component and an optical waveguide which is optically connected to the light source and inside which the light from the light source propagates and at least a portion of which is exposed to a space within the acoustic cavity.

### Item 2

The optical concentration measuring device according to item 1, wherein
the acoustic transducer outputs an electrical signal according to a pressure fluctuation within the acoustic cavity that is generated by causing the target component included in the gas that is present around the optical waveguide and having a lower refractive index than the optical waveguide to absorb an evanescent wave that leaks to an outside of the optical waveguide during propagation of the light from the light source within the optical waveguide.

### Item 3

The optical concentration measuring device according to item 1, wherein
the optical concentration measuring device has a volume of 10 mm³ or less, and an effective optical path length of the optical waveguide is 10 mm or more.

### Item 4

The optical concentration measuring device according to any one of items 1 to 3, wherein
the optical part has one or more substrates that support the light source and the optical waveguide.

### Item 5

An optical concentration measuring device according to item 4, wherein
the light source and the optical waveguide are provided together on one substrate, and are in direct contact with each other.

### Item 6

The optical concentration measuring device according to item 4, wherein
the light source and the optical waveguide are provided together on one substrate, and are optically coupled to each other via a photocoupler.

### Item 7

The optical concentration measuring device according to item 4, wherein
the light source and the optical waveguide are respectively provided on separate substrates of the one or more substrates, and are optically coupled to each other via a photocoupler.

### Item 8

The optical concentration measuring device according to item 4, wherein
an entire surface of a section of at least a portion of the optical waveguide in a propagation direction of the light that propagates within the optical waveguide is exposed to a space within the acoustic cavity without being in contact with the substrate.

### Item 9

The optical concentration measuring device according to item 4, wherein
a space is provided between the light source and the substrate.

### Item 10

The optical concentration measuring device according to any one of items 1 to 3, wherein
the optical waveguide includes a bending portion that bends, at least in part, a propagation direction of the light that propagates within the optical waveguide.

### Item 11

The optical concentration measuring device according to any one of items 1 to 3, wherein
the optical waveguide includes one or more pairs of reflecting means that, at least in part, reverse and thereafter restore a propagation direction of the light that propagates within the optical waveguide.

### Item 12

The optical concentration measuring device according to any one of items 1 to 3, wherein
the optical waveguide includes an elliptic circulating portion that causes, at least in part, the light that propagates within the optical waveguide to circulate in an elliptical manner.

### Item 13

The optical concentration measuring device according to any one of items 1 to 3, wherein
the optical waveguide includes a ring-shaped circulating portion that causes, at least in part, the light that propagates within the optical waveguide to circulate in an elliptical or circular manner.

### Item 14

The optical concentration measuring device according to any one of items 1 to 3, wherein
the optical part an optical receiver that detects the light from the light source, and
the light source adjusts an output of the light to be emitted according to a detection result of the optical receiver.

### Item 15

The optical concentration measuring device according to any one of items 1 to 3, wherein
the optical part has an optical filter that transmits the wavelength, among the light from the light source, and shields wavelengths other than the wavelength.

### Item 16

The optical concentration measuring device according to any one of items 1 to 3, wherein
the optical part has a plurality of optical waveguides, each being identical to the optical waveguide, that are optically connected to one light source.

### Item 17

The optical concentration measuring device according to any one of items 1 to 3, wherein
the optical part has a plurality of the light sources optically connected to one or more of the optical waveguides.

### Item 18

The optical concentration measuring device according to any one of items 1 to 3, wherein
an average optical path length of light, among the light from the light source, that is emitted, without entering the optical waveguide, to a space within the acoustic cavity and reflected by an inner wall of the acoustic cavity is shorter than an effective optical path length of the optical waveguide.

### Item 19

The optical concentration measuring device according to any one of items 1 to 3,
wherein an average reflectance of an inner wall of the acoustic cavity is 70% or less.

### Item 20

An optical concentration measuring device according to any one of items 1 to 3, wherein the acoustic cavity has:
a foundation portion on which the optical part and the acoustic transducer are installed on a surface thereof; and
a lid portion on which the gas inlet opening is formed and which is attached to the foundation portion.

### Item 21

The optical concentration measuring device according to item 20, wherein
the lid portion is adhered to the foundation portion, and thereby an internal space of the acoustic cavity communicates to an external space of the acoustic cavity only via the gas inlet opening.

### Item 22

The optical concentration measuring device according to item 20, wherein
the foundation portion has a conductive portion, and
the conductive portion includes:
an inner contact end that is formed on the surface and that is electrically connected to at least any of the light source or the acoustic transducer; and
an outer contact end that is exposed to an external space of the acoustic cavity.

### Item 23

The optical concentration measuring device according to any one of items 1 to 3, comprising:
a dustproof filter that is attached to the gas inlet opening to seal the gas contained within the acoustic cavity.

### Item 24

The optical concentration measuring device according to any one of items 1 to 3, comprising:
an electrical circuit that is installed within the acoustic cavity and electrically connected to at least any of the light source or the acoustic transducer.

### EXPLANATION OF REFERENCES

100: optical concentration measuring device,
110: acoustic cavity,
111: gas inlet opening,
120: optical part,
130: light source,
140: optical waveguide,
150: substrate,
190: acoustic transducer,
220: optical part,
300: optical concentration measuring device,
320: optical part,
360: photocoupler,
321: optical part,
361: photocoupler,
322: optical part,
362: photocoupler,
323: optical part,
363: photocoupler,
400: optical concentration measuring device,
420: optical part,
451: substrate,
452: substrate,
460: photocoupler,
401: optical concentration measuring device,
421: optical part,
453: substrate support,
402: optical concentration measuring device,
422: optical part,
500: optical concentration measuring device,
520: optical part,
550: substrate,
551: supporting portion,
600: optical concentration measuring device,
620: optical part,
650: substrate,
651: supporting portion,
621: optical part,
622: optical part,
640: optical waveguide,
741: optical waveguide,
742: optical waveguide,
743: optical waveguide,
820: optical part,
840: optical waveguide,
841: reflecting means,
920: optical part,
921: optical part,
940: optical waveguide,
941: elliptic circulating portion,
950: substrate,
951: supporting portion,
1021: optical part,
1130: light source,
1120: optical part,
1140: optical waveguide,
1141: ring-shaped circulating portion,
1220: optical part,
1240: optical waveguide,
1241: ring-shaped circulating portion,
1300: optical concentration measuring device,
1320: optical part,
1370: optical receiver,
1400: optical concentration measuring device,
1420: optical part,
1450: substrate,
1451: supporting portion,
1452: substrate,
1470: optical receiver,
1520: optical part,
1570: optical receiver,
1620: optical part,
1670: optical receiver,
1800: optical concentration measuring device,
1820: optical part,
1880: optical filter,
1821: optical part,
1831: light source,
1881: optical filter,
1822: optical part,
1832: light source,
1882: optical filter,
1823: optical part,
1833: light source,
1883: optical filter,
1900: optical concentration measuring device,
1920: optical part,
1940: optical waveguide,
1901: optical concentration measuring device,
1921: optical part,
1941: optical waveguide,
2000: optical concentration measuring device,
2020: optical part,
2030: light source,
2040: optical waveguide,
2001: optical concentration measuring device,
2021: optical part,
2031: light source,
2041: optical waveguide,
2100: optical concentration measuring device,
2110: acoustic cavity,
2112: foundation portion,
2113: surface,
2114: lid portion,
2115: adhesive
2200: optical concentration measuring device,
2210: acoustic cavity,
2212: foundation portion,
2213: surface,
2216: conductive portion,
2217: inner contact end,
2218: outer contact end,
2300: optical concentration measuring device,
2392: dustproof filter,
2400: optical concentration measuring device,
2494: electrical circuit.

What is claimed is:

1. An optical concentration measuring device for measuring a concentration of a target component in a gas, the optical concentration measuring device comprising:
an acoustic cavity that has a gas inlet opening and contains the gas introduced therein via the gas inlet opening;
an optical part installed within the acoustic cavity; and
an acoustic transducer installed within the acoustic cavity,
wherein the optical part includes one or more of each of a light source that emits light including a wavelength that is absorbed by the target component and an optical waveguide which is optically connected to the light source and inside which the light from the light source propagates and at least a portion of which is exposed to a space within the acoustic cavity, and
the acoustic transducer outputs an electrical signal according to a pressure fluctuation within the acoustic cavity that is generated by causing the target component included in the gas that is present around the optical waveguide and having a lower refractive index than the optical waveguide to absorb an evanescent wave that leaks to an outside of the optical waveguide during propagation of the light from the light source within the optical waveguide.

2. The optical concentration measuring device according to claim 1,
wherein the optical part has one or more substrates that support the light source and the optical waveguide, and
the light source and the optical waveguide are provided together on one substrate, and are optically coupled to each other via a photocoupler.

3. The optical concentration measuring device according to claim 1,
wherein the optical part has one or more substrates that support the light source and the optical waveguide, and
the light source and the optical waveguide are respectively provided on separate substrates of the one or more substrates, and are optically coupled to each other via a photocoupler.

4. The optical concentration measuring device according to claim 1,
wherein the optical part has one or more substrates that support the light source and the optical waveguide, and
a space is provided between the light source and the substrate.

5. The optical concentration measuring device according to claim 1,
wherein the optical waveguide includes a bending portion that bends, at least in part, a propagation direction of the light that propagates within the optical waveguide.

6. The optical concentration measuring device according to claim 1,
wherein the optical waveguide includes one or more pairs of reflecting means that, at least in part, reverse and thereafter restore a propagation direction of the light that propagates within the optical waveguide.

7. The optical concentration measuring device according to claim 1,
wherein the optical waveguide includes an elliptic circulating portion that causes, at least in part, the light that propagates within the optical waveguide to circulate in an elliptical manner.

8. The optical concentration measuring device according to claim 1,
wherein the optical waveguide includes a ring-shaped circulating portion that causes, at least in part, the light that propagates within the optical waveguide to circulate in an elliptical or circular manner.

9. The optical concentration measuring device according to claim 1, wherein the optical part has an optical filter that transmits the wavelength, among the light from the light source, and shields wavelengths other than the wavelength.

10. The optical concentration measuring device according to claim 1,
wherein the optical part has a plurality of optical waveguides, each being identical to the optical waveguide, that are optically connected to one light source.

11. The optical concentration measuring device according to claim 1,
wherein the optical part has a plurality of light sources, each being identical to the light source, that are optically connected to one or more optical waveguides, each being identical to the optical waveguide.

12. The optical concentration measuring device according to claim 1,
wherein an average optical path length of light, among the light from the light source, that is emitted, without entering the optical waveguide, to a space within the acoustic cavity and reflected by an inner wall of the acoustic cavity is shorter than an effective optical path length of the optical waveguide.

13. The optical concentration measuring device according to claim 1, wherein
the optical concentration measuring device has a volume of 10 mm³ or less, and
an effective optical path length of the optical waveguide is 10 mm or more.

14. The optical concentration measuring device according to claim 1,
wherein an average reflectance of an inner wall of the acoustic cavity is 70% or less.

15. The optical concentration measuring device according to claim 1, wherein
the acoustic cavity has:
a foundation portion on which the optical part and the acoustic transducer are installed on a surface thereof; and
a lid portion on which the gas inlet opening is formed and which is attached to the foundation portion.

16. The optical concentration measuring device according to claim 15, wherein
the lid portion is adhered to the foundation portion, and thereby an internal space of the acoustic cavity communicates to an external space of the acoustic cavity only via the gas inlet opening.

17. The optical concentration measuring device according to claim 15, wherein
the foundation portion has a conductive portion, and
the conductive portion includes:
an inner contact end that is formed on the surface and that is electrically connected to at least any of the light source or the acoustic transducer; and
an outer contact end that is exposed to an external space of the acoustic cavity.

18. The optical concentration measuring device according to claim 1, comprising:
a dustproof filter that is attached to the gas inlet opening to seal the gas contained within the acoustic cavity.

19. The optical concentration measuring device according to claim 1, comprising:
an electrical circuit that is installed within the acoustic cavity and electrically connected to at least any of the light source or the acoustic transducer.
